(19) United States
(12) Patent Application Publication
(10) Pub. No.: US 2014/0170653 A1
(43) Pub. Date: Jun. 19, 2014

(54) CHEMICAL LIGATION

(71) Applicant: Lai-Qiang Ying, Eugene, OR (US)

(72) Inventor: Lai-Qiang Ying, Eugene, OR (US)

(73) Assignee: Life Technologies Corporation, Carlsbad, CA (US)

(21) Appl. No.: 14/111,675

(22) PCT Filed: Apr. 9, 2012
(86) PCT No.: PCT/US12/32802

§ 371 Date: Feb. 28, 2014

Related U.S. Application Data
(60) Provisional application No. 61/476,130, filed on Apr. 15, 2011.

(51) Int. Cl. C12Q 1/68

(52) U.S. Cl. CPC C12Q 1/6883
USPC 435/6.11; 536/24.31

(57) ABSTRACT

Methods comprising chemical ligation of oligonucleotides are provided. In some embodiments, methods of detecting a polymorphisms in nucleic acids are provided. In some embodiments, methods of detecting at least one analyte are provided. In some embodiments, methods of labeling solid support particles are provided. Kits comprising oligonucleotides with chemically ligatable moieties are also provided.

### BACKGROUND

Chemical ligation allows covalent ligation between two oligonucleotides in the absence of ligase. Various template-dependent chemical ligation methods have been described, including ligation between two peptide nucleic acid (PNA) oligonucleotides using carbodiimide coupling reagents, various metal-mediated ligation methods, and reagent-free methods. Reagent-free methods generally involve the use of a nucleophile on the first oligonucleotide and an electrophile on the second oligonucleotide. For example, chemical ligation between an oligonucleotide with a 5′ bromoacetyl group and an oligonucleotide with a 3′ phosphorothioate group have previously been demonstrated. This reagent-free chemical ligation method was later modified by replacing the 5′ bromoacetyl group with a 5′ tosyl or 5′ iodide.

Chemical ligation allows the use of ligation under conditions that may not be suitable for an enzyme, and eliminates the expense of using a ligase.

### SUMMARY

In some embodiments, methods of detecting a single nucleotide polymorphism in a target nucleic acid are provided. In some embodiments, the method comprises (a) contacting said target nucleic acid with a first allele-specific primer that hybridizes to a portion of the target nucleic acid comprising the single nucleotide polymorphism and a locus-specific primer, wherein the first allele-specific primer comprises a 3′ nucleophile, and the locus-specific primer comprises a 5′ leaving group, wherein the first allele specific primer and the locus-specific primer hybridize to the target nucleic acid such that the 5′ end of the locus-specific primer is adjacent to the 3′ end of the first allele-specific primer, under conditions allowing chemical ligation between the first allele-specific primer and the first locus-specific primer to form a ligated product; and (b) detecting the ligated product. In some embodiments, the method further comprises contacting the target nucleic acid with a second allele-specific primer that hybridizes to a portion of the target nucleic acid comprising the single nucleotide polymorphism, wherein the second allele-specific primer comprises a 3′ nucleophile, wherein the second allele-specific primer differs from the first allele-specific primer at least at the nucleotide that hybridizes with the single nucleotide polymorphism, and wherein the second allele specific primer and the locus-specific primer hybridize to the target nucleic acid such that the 5′ end of the locus-specific primer is adjacent to the 3′ end of the second allele-specific primer.

In some embodiments, a method of detecting a single nucleotide polymorphism in a target nucleic acid comprises (a) contacting the target nucleic acid with a first allele-specific primer that hybridizes to a portion of the target nucleic acid comprising the single nucleotide polymorphism and a locus-specific primer, wherein the first allele-specific primer comprises a 5′ leaving group, and the locus-specific primer comprises a 3′ nucleophile, wherein the first allele-specific primer and the locus-specific primer hybridize to the target nucleic acid such that the 5′ end of the first allele-specific primer is adjacent to the 3′ end of the locus-specific primer, under conditions allowing chemical ligation between the first allele-specific primer and the locus-specific primer to form a ligated product; and (b) detecting the ligated product. In some embodiments, the method further comprises contacting the target nucleic acid with a second allele-specific primer that hybridizes to a portion of the target nucleic acid comprising the single nucleotide polymorphism, wherein the second allele-specific primer comprises a 5′ leaving group, wherein the second allele-specific primer differs from the first allele-specific primer at least at the nucleotide that hybridizes with the single nucleotide polymorphism, and wherein the second allele-specific primer and the locus-specific primer hybridize to the target nucleic acid such that the 5′ end of the second allele-specific primer is adjacent to the 3′ end of the locus-specific primer.

In some embodiments, the locus-specific primer comprises a sequence that is complementary to between 3 and 60 contiguous nucleotides of the target nucleic acid. In some embodiments, the first allele-specific primer comprises a sequence that is complementary to between 3 and 60 contiguous nucleotides of the target nucleic acid. In some embodiments, the second allele-specific primer comprises a sequence that is complementary to between 3 and 60 contiguous nucleotides of the target nucleic acid.

In some embodiments, detecting the ligated product comprises enzymatically amplifying the ligated product. In some embodiments, detecting the ligated product comprises enzymatically amplifying the ligated product using one or more of real-time PCR, qPCR, and digital PCR. In some embodiments, the first allele-specific primer comprises a first portion that is complementary to the target nucleic acid and a second portion that is complementary to a first amplification primer, and the locus-specific primer comprises a first portion that is complementary to the target nucleic acid and a second portion that is complementary to a second amplification primer, and wherein detecting the ligated product comprises enzymatically amplifying the ligated product in the presence of the first amplification primer and the second amplification primer.

In some embodiments, the first allele-specific primer comprises a detectable label, or the locus-specific primer comprises a detectable label, or the first allele-specific primer comprises a first detectable label and the locus-specific primer comprises a second detectable label, wherein the first and second detectable labels are the same or different.

In some embodiments, methods of detecting at least one target analyte in a sample are provided. In some such embodiments, a method comprises (a) contacting the at least one target analyte with: (i) a first proximity detection probe comprising a first analyte binding moiety and a first oligonucleotide moiety, wherein the first oligonucleotide moiety comprises a 3′ nucleophile; (ii) a second proximity detection probe comprising a second analyte binding moiety and a second oligonucleotide moiety, wherein the second oligonucleotide moiety comprises a 5′ leaving group; and (iii) a splint oligonucleotide comprising a first portion that hybridizes with a portion of the first oligonucleotide moiety and a second portion that hybridizes with the second oligonucleotide moiety such that the 3′ end of the first oligonucleotide moiety is adjacent to the 5′ end of the second oligonucleotide moiety; under conditions allowing formation of a complex comprising at least one target analyte, the first proximity detection probe, the second proximity detection probe, and the splint oligonucleotide, and allowing chemical ligation between the first oligonucleotide moiety and the second oligonucleotide moiety to form a ligated product; and (b) detecting the ligated product.

In some embodiments, the method comprises removing unbound first proximity detection probe, removing unbound second proximity detection probe, or removing unbound first proximity detection probe and removing unbound second proximity detection probe.

In some embodiments, the target analyte is selected from a protein, a peptide, a carbohydrate, and a hormone. In some embodiments, the target analyte is a protein. In some embodiments, the first analyte binding moiety and the second analyte binding moiety are capable of binding to the same target analyte. In some embodiments, the first analyte binding moiety and the second analyte binding moiety are capable of binding to different target analytes.

In some embodiments, at least one of the analyte binding moieties is a covalent analyte binding moiety. In some embodiments, the covalent analyte binding moiety is capable of covalently attaching to an enzyme selected from a metalloprotease, a cysteine protease, a ubiquitin-specific protease, a cysteine cathepsin, an esterase, a kinase, a histone deacetylase, a serine reductase, an oxidoreductase, an ATPase, and a GTPase. In some embodiments, at least one of the analyte binding moieties is a noncovalent analyte binding moiety. In some embodiments, the noncovalent analyte binding moiety is selected from an antibody, a protein, a peptide, a lectin, a nucleic acid, an aptamers, a carbohydrate, a soluble receptor, and a small molecule.

In some embodiments, the detecting comprises enzymatically amplifying the ligated product. In some embodiments, the detecting comprises enzymatically amplifying the ligated product using one or more of real-time PCR, qPCR, and digital PCR.

In some embodiments, methods of labeling solid support particles are provided. In some such embodiments, a method comprises contacting a solid support particle comprising a first member of a binding pair, with: (i) a first oligonucleotide moiety comprising a 3′ nucleophile, and further comprising a second member of a binding pair; (ii) a second oligonucleotide moiety comprising a 5′ leaving group, and further comprising at least one detectable label; and (iii) a splint oligonucleotide comprising a first portion that hybridizes with a portion of the first oligonucleotide moiety and a second portion that hybridizes with the second oligonucleotide moiety such that the 3′ end of the first oligonucleotide moiety is adjacent to the 5′ end of the second oligonucleotide moiety; under conditions allowing binding of the first member of the binding pair to the second member of the binding pair, and allowing formation of a complex comprising the solid support particle, the first oligonucleotide moiety, the second oligonucleotide moiety, and the splint oligonucleotide, and allowing chemical ligation between the first oligonucleotide moiety and the second oligonucleotide moiety.

In some embodiments, a method of labeling a solid support particle comprises combining a solid support particle comprising a first member of a binding pair, with: (i) a first oligonucleotide moiety comprising a 5′ leaving group, and further comprising a second member of a binding pair; (ii) a second oligonucleotide moiety comprising a 3′ nucleophile, and further comprising at least one detectable label; and (iii) a splint oligonucleotide comprising a first portion that hybridizes with a portion of the first oligonucleotide moiety and a second portion that hybridizes with the second oligonucleotide moiety such that the 5′ end of the first oligonucleotide moiety is adjacent to the 3′ end of the second oligonucleotide moiety; under conditions allowing binding of the first member of the binding pair to the second member of the binding pair, and allowing formation of a complex comprising the solid support particle, the first oligonucleotide moiety, the second oligonucleotide moiety, and the splint oligonucleotide, and allowing chemical ligation between the first oligonucleotide moiety and the second oligonucleotide moiety.

In some embodiments, the ratio of second oligonucleotide to first oligonucleotide is between 10:1 and 1:200, between 5:1 and 1:100, or between 2:1 and 1:50. In some embodiments, the ratio of splint oligonucleotide and first oligonucleotide is between 10:1 and 1:200, between 5:1 and 1:100, or between 2:1 and 1:50.

In some embodiments, a method of labeling a solid support particle comprises contacting a solid support particle comprising a first member of a binding pair, with: (i) a first oligonucleotide moiety comprising a 3′ nucleophile, and further comprising a second member of a binding pair; (ii) a second oligonucleotide moiety comprising a 5′ leaving group, and further comprising a first detectable label; (iii) a third oligonucleotide moiety comprising a 5′ leaving group, and further comprising a second detectable label; (iv) a first splint oligonucleotide comprising a first portion that hybridizes with a portion of the first oligonucleotide moiety and a second portion that hybridizes with the second oligonucleotide moiety such that the 3′ end of the first oligonucleotide moiety is adjacent to the 5′ end of the second oligonucleotide moiety; and (iv) a second splint oligonucleotide comprising a first portion that hybridizes with a portion of the first oligonucleotide moiety and a second portion that hybridizes with the third oligonucleotide moiety such that the 3′ end of the first oligonucleotide moiety is adjacent to the 5′ end of the third oligonucleotide moiety; under conditions allowing binding of the first member of the binding pair to the second member of the binding pair, and allowing formation of a first complex comprising the solid support particle, the first oligonucleotide moiety, the second oligonucleotide moiety, and the first splint oligonucleotide, and a second complex comprising the solid support particle, the first oligonucleotide moiety, the third oligonucleotide moiety, and the second splint oligonucleotide, and allowing chemical ligation between the first oligonucleotide moiety and the second oligonucleotide moiety, and between the first oligonucleotide moiety and the third oligonucleotide moiety.

In some embodiments, the ratio of first splint oligonucleotide to second splint oligonucleotide is between 500:1 and 1:500, between 100:1 and 1:100, or between 10:1 and 1:10.

In some embodiments, the method further comprises combining the solid particle comprising a first member of a binding pair with: (v) a fourth oligonucleotide moiety comprising a 5′ leaving group, and further comprising a third detectable label; and (vi) a third splint oligonucleotide comprising a first portion that hybridizes with a portion of the first oligonucleotide moiety and a second portion that hybridizes with the fourth oligonucleotide moiety such that the 3′ end of the first oligonucleotide moiety is adjacent to the 5′ end of the fourth oligonucleotide moiety; under conditions allowing binding of the first member of the binding pair to the second member of the binding pair, and allowing formation of a complex comprising the solid support particle, the first oligonucleotide moiety, the fourth oligonucleotide moiety, and the third splint oligonucleotide, and allowing chemical ligation between the first oligonucleotide moiety and the fourth oligonucleotide moiety.

In some embodiments, the method further comprises combining the solid particle comprising a first member of a binding pair with: (v) a fifth oligonucleotide moiety comprising a 5′ leaving group, and further comprising a fourth detectable label; and (vi) a fourth splint oligonucleotide comprising a first portion that hybridizes with a portion of the first oligonucleotide moiety and a second portion that hybridizes with the fifth oligonucleotide moiety such that the 3′ end of the first oligonucleotide moiety is adjacent to the 5′ end of the fifth oligonucleotide moiety; under conditions allowing binding of the first member of the binding pair to the second member of the binding pair, and allowing formation of a complex comprising the solid support particle, the first oligonucleotide moiety, the fifth oligonucleotide moiety, and the fourth splint oligonucleotide, and allowing chemical ligation between the first oligonucleotide moiety and the fifth oligonucleotide moiety.

In some embodiments, a method of labeling a solid support particle comprises contacting a solid support particle comprising a first member of a binding pair, with: (i) a first oligonucleotide moiety comprising a 5′ leaving group, and further comprising a second member of a binding pair; (ii) a second oligonucleotide moiety comprising a 3′ nucleophile, and further comprising a first detectable label; (iii) a third oligonucleotide moiety comprising a 3′ nucleophile, and further comprising a second detectable label; (iv) a first splint oligonucleotide comprising a first portion that hybridizes with a portion of the first oligonucleotide moiety and a second portion that hybridizes with the second oligonucleotide moiety such that the 5′ end of the first oligonucleotide moiety is adjacent to the 3′ end of the second oligonucleotide moiety; and (iv) a second splint oligonucleotide comprising a first portion that hybridizes with a portion of the first oligonucleotide moiety and a second portion that hybridizes with the third oligonucleotide moiety such that the 5′ end of the first oligonucleotide moiety is adjacent to the 3′ end of the third oligonucleotide moiety; under conditions allowing binding of the first member of the binding pair to the second member of the binding pair, and allowing formation of a first complex comprising the solid support particle, the first oligonucleotide moiety, the second oligonucleotide moiety, and the first splint oligonucleotide, and a second complex comprising the solid support particle, the first oligonucleotide moiety, the third oligonucleotide moiety, and the second splint oligonucleotide, and allowing chemical ligation between the first oligonucleotide moiety and the second oligonucleotide moiety, and between the first oligonucleotide moiety and the third oligonucleotide moiety.

In some embodiments, the ratio of first splint oligonucleotide to second splint oligonucleotide is between 500:1 and 1:500, between 100:1 and 1:100, or between 10:1 and 1:10.

In some embodiments, the method further comprises combining the solid particle comprising a first member of a binding pair with: (v) a fourth oligonucleotide moiety comprising a 3′ nucleophile, and further comprising a third detectable label; and (vi) a third splint oligonucleotide comprising a first portion that hybridizes with a portion of the first oligonucleotide moiety and a second portion that hybridizes with the fourth oligonucleotide moiety such that the 5′ end of the first oligonucleotide moiety is adjacent to the 3′ end of the fourth oligonucleotide moiety; under conditions allowing binding of the first member of the binding pair to the second member of the binding pair, and allowing formation of a complex comprising the solid support particle, the first oligonucleotide moiety, the fourth oligonucleotide moiety, and the third splint oligonucleotide, and allowing chemical ligation between the first oligonucleotide moiety and the fourth oligonucleotide moiety.

In some embodiments, the method further comprises combining the solid particle comprising a first member of a binding pair with: (v) a fifth oligonucleotide moiety comprising a 3′ nucleophile, and further comprising a fourth detectable label; and (vi) a fourth splint oligonucleotide comprising a first portion that hybridizes with a portion of the first oligonucleotide moiety and a second portion that hybridizes with the fifth oligonucleotide moiety such that the 5′ end of the first oligonucleotide moiety is adjacent to the 3′ end of the fifth oligonucleotide moiety; under conditions allowing binding of the first member of the binding pair to the second member of the binding pair, and allowing formation of a complex comprising the solid support particle, the first oligonucleotide moiety, the fifth oligonucleotide moiety, and the fourth splint oligonucleotide, and allowing chemical ligation between the first oligonucleotide moiety and the fifth oligonucleotide moiety.

In some embodiments, the first member of the binding pair is a biotin-binding moiety and the second member of the binding pair is biotin or a biotin derivative. In some embodiments, the biotin-binding moiety is avidin or streptavidin.

In some embodiments, a 3′ nucleophile is selected from phosphorothioate, phosphoroselenoate, phosphorotelluroate, thiol, thiocarboxylate, dithiocarboxylate, amino, hydrazine, hydroxylamine, selenol, selenocarboxylate, and diselenocarboxylate. In some embodiments, the 3′ nucleophile is selected from phosphorothioate, phosphoroselenoate, and phosphorotelluroate.

In some embodiments, the 5′ leaving group is selected from I, Br, Cl, mesylate, tosylate, brosylate, para-nitrobenzenesulfonate, trifluoromethanesulfonate, trifluoroethanesulfonate, nonafluorobutanesulfonate, trifluoroacetate, a sulfonium cation, and a quaternary ammonium cation. In some embodiments, the 5′ leaving group is selected from I, Br, and tosylate.

In some embodiments, kits are provided. In some embodiments, a kit comprises a first allele-specific primer and a locus-specific primer, wherein the first allele-specific primer comprises a 3′ nucleophile, and the locus-specific primer comprises a 5′ leaving group, wherein the first allele specific primer hybridizes to a portion of a target nucleic acid comprising a single nucleotide polymorphism, and wherein the first allele-specific primer and the locus-specific primer hybridize to a target nucleic acid such that the 5′ end of the locus-specific primer is adjacent to the 3′ end of the first allele-specific primer. In some embodiments, a kit further comprises a second allele-specific primer, wherein the second allele-specific primer comprises a 3′ nucleophile, wherein the second allele-specific primer differs from the first allele-specific primer at least at the nucleotide that hybridizes with the single nucleotide polymorphism, and wherein the second allele specific primer and the locus-specific primer hybridize to the target nucleic acid such that the 5′ end of the locus-specific primer is adjacent to the 3′ end of the second allele-specific primer.

In some embodiments, a kit comprises a first allele-specific primer and a locus-specific primer, wherein the first allele-specific primer comprises a 5′ leaving group, and the locus-specific primer comprises a 3′ nucleophile, wherein the first allele specific primer hybridizes to a portion of a target nucleic acid comprising a single nucleotide polymorphism, and wherein the first allele-specific primer and the locus-specific primer hybridize to a target nucleic acid such that the 3′ end of the locus-specific primer is adjacent to the 5′ end of the first allele-specific primer. In some embodiments, a kit further comprises a second allele-specific primer, wherein the second allele-specific primer comprises a 5′ leaving group, wherein the second allele-specific primer differs from the first allele-specific primer at least at the nucleotide that hybridizes with the single nucleotide polymorphism, and wherein the second allele specific primer and the locus-specific primer hybridize to the target nucleic acid such that the 3′ end of the locus-specific primer is adjacent to the 5′ end of the second allele-specific primer.

In some embodiments, a kit comprises a first proximity detection probe comprising a first analyte binding moiety and a first oligonucleotide moiety, wherein the first oligonucleotide moiety comprises a 3′ nucleophile; and a second proximity detection probe comprising a second analyte binding moiety and a second oligonucleotide moiety, wherein the second oligonucleotide moiety comprises a 5′ leaving group. In some embodiments, a kit further comprises a splint oligonucleotide comprising a first portion that hybridizes with a portion of the first oligonucleotide moiety and a second portion that hybridizes with the second oligonucleotide moiety such that the 3′ end of the first oligonucleotide moiety is adjacent to the 5′ end of the second oligonucleotide moiety.

In some embodiments, the first analyte binding moiety and the second analyte binding moiety are capable of binding to the same target analyte. In some embodiments, the first analyte binding moiety and the second analyte binding moiety are capable of binding to different target analytes. In some embodiments, at least one of the analyte binding moieties is a covalent analyte binding moiety. In some embodiments, the covalent analyte binding moiety is capable of covalently attaching to an enzyme selected from a metalloprotease, a cysteine protease, a ubiquitin-specific protease, a cysteine cathepsin, an esterase, a kinase, a histone deacetylase, a serine reductase, an oxidoreductase, an ATPase, and a GTPase. In some embodiments, at least one of the analyte binding moieties is a noncovalent analyte binding moiety. In some embodiments, the noncovalent analyte binding moiety is selected from an antibody, a protein, a peptide, a lectin, a nucleic acid, an aptamers, a carbohydrate, a soluble receptor, and a small molecule.

In some embodiments, the 3′ nucleophile is selected from phosphorothioate, phosphoroselenoate, phosphorotelluroate, thiol, thiocarboxylate, dithiocarboxylate, amino, hydrazine, hydroxylamine, selenol, selenocarboxylate, and diselenocarboxylate. In some embodiments, the 3′ nucleophile is selected from phosphorothioate, phosphoroselenoate, and phosphorotelluroate. In some embodiments, the 5′ leaving group is selected from I, Br, Cl, mesylate, tosylate, brosylate, para-nitrobenzenesulfonate, trifluoromethanesulfonate, trifluoroethanesulfonate, nonafluorobutanesulfonate, trifluoroacetate, a sulfonium cation, and a quaternary ammonium cation. In some embodiments, the 5′ leaving group is selected from I, Br, and tosylate.

### BRIEF DESCRIPTION OF FIGURES

FIG. 1 shows (A) a graph of the rate and specificity of chemical ligation between a FAM-labeled oligonucleotide containing a 3′-phosphorothioate and a biotin-labeled oligonucleotide containing a 5′-iodo leaving group in the presence of a DNA template at 37° C. and 50° C., and (B) a graph of the rate and specificity of chemical ligation between a FAM-labeled oligonucleotide containing a 3′-phosphorothioate and a biotin-labeled oligonucleotide containing a 5′-iodo leaving group in the presence of an RNA template at 50° C., as described in Example 1.

FIG. 2A shows detection sensitivity of ligated product using enzymatic ligation or chemical ligation between a FAM-labeled oligonucleotide and a biotin-labeled oligonucleotide in the presence of a DNA template, as described in Example 2. Panels B and C show the ligation signal and control signal detected following ligation using flow cytometry for enzymatic ligation and chemical ligation, respectively.

FIG. 3 shows the selectivity of chemical ligation between FAM-labeled oligonucleotides with single base mismatches at various positions, and a biotin-labeled oligonucleotide, in the presence of a DNA template at 37° C. and 50° C., as described in Example 3.

FIG. 4 shows the selectivity of enzymatic ligation between FAM-labeled oligonucleotides with A, C, G, or T at the 3′ terminus and a biotin-labeled oligonucleotide in the presence of DNA templates with C, T, A, or G at the site that hybridizes with the 3′ terminus of the FAM-labeled oligonucleotides, as described in Example 3.

FIG. 5A-D show the selectivity of chemical ligation between FAM-labeled oligonucleotides with G, A, T, or C at the 3′ terminus and a biotin-labeled oligonucleotide in the presence of DNA templates with C, T, A, or G at the site that hybridizes with the 3′ terminus of the FAM-labeled oligonucleotides, as described in Example 3.

FIG. 6 shows signal amplification of a chemical ligation product under isothermal or thermalcycled amplification conditions at various concentrations of DNA template, with a ligation temperature of either 37° C. or 50° C., as described in Example 4.

FIG. 7 shows bar graphs of (A) chemical ligation yield at 45° C. and 50° C. between a FAM-labeled oligonucleotide with a 3′-terminal A, a VIC-labeled oligonucleotide with a 3′-terminal G, and a biotin-labeled oligonucleotide, in the presence various ratios of two DNA templates, one with a C at the site that hybridizes with the 3′ terminus of the labeled oligonucleotides, and one with a T at that site, and (B) chemical ligation yield at 45° C. and 50° C. between a FAM-labeled oligonucleotide with a 3′-terminal T, a VIC-labeled oligonucleotide with a 3′-terminal C, and a biotin-labeled oligonucleotide, in the presence various ratios of two DNA templates, one with a A at the site that hybridizes with the 3′ terminus of the labeled oligonucleotides, and one with a G at that site, as described in Example 5.

FIG. 8 shows a bar graph of relative intensity of FAM labeling when different ratios of FAM-labeled oligonucleotide (“probe oligo”) to biotin-labeled oligonucleotide (“capture oligo”) are chemically ligated in the presence of a DNA template to label a solid support particle, as described in Example 6.

FIG. 9 shows a bar graph of relative intensity of FAM labeling when different ratios of DNA template (“template”) to biotin-labeled oligonucleotide (“capture oligo”) are used in a chemical ligation between the biotin-labeled oligonucleotide and a FAM-labeled oligonucleotide to label a solid support particle, as described in Example 6.

FIG. 10 shows a bar graph of relative intensity of two labels, FAM and VIC, when a mixture of FAM-labeled oligonucleotide with a C at the 3′ terminus and VIC-labeled oligonucleotide with a G at the 3′ terminus are chemically ligated to a biotin-labeled oligonucleotide in the presence of various ratios of DNA templates, one with a G at the site that hybridizes with the 3′ terminus of the labeled oligonucleotides, and one with a C at that site, to label a solid support particle, as described in Example 7.

FIG. 11 shows a scheme for encoding solid support particles with four different labels in varying ratios, as described herein.

### DETAILED DESCRIPTION

Methods comprising chemical ligation of oligonucleotides are provided. In some embodiments, methods of detecting polymorphisms in nucleic acids are provided. In some embodiments, methods of detecting at least one analyte are provided. In some embodiments, methods of labeling solid support particles are provided. Kits comprising oligonucleotides with chemically ligatable moieties are also provided.

The section headings used herein are for organizational purposes only and are not to be construed as limiting the subject matter described.

### DEFINITIONS

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

Exemplary techniques used in connection with recombinant DNA, oligonucleotide synthesis, tissue culture, enzymatic reactions, and purification are known in the art. Many such techniques and procedures are described, e.g., in Sambrook et al. Molecular Cloning: A Laboratory Manual (2nd ed., Cold Spring Harbor Laboratory Press, Cold Spring Harbor, N.Y. (1989)), among other places. In addition, exemplary techniques for chemical syntheses are also known in the art.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As utilized in accordance with the present disclosure, the following terms, unless otherwise indicated, shall be understood to have the following meanings:

The terms “nucleic acid” and “polynucleotide” may be used interchangeably, and refer to a polymer of nucleotides. Such polymers of nucleotides may contain natural and/or non-natural nucleotides, and include, but are not limited to, DNA, RNA, PNA, LNA and any other nucleotide polymer that can be ligated and is PCR competent. “Nucleic acid sequence” or “polynucleotide sequence” may be used interchangeably, and refer to the linear sequence of nucleotides in the nucleic acid or polynucleotide.

The terms “annealing” and “hybridizing” are used interchangeably and refer to the base-pairing interaction of one nucleic acid with another nucleic acid that results in formation of a duplex, triplex, or other higher-ordered structure. In some embodiments, the primary interaction is base specific, e.g., A/T and G/C, by Watson/Crick and Hoogsteen-type hydrogen bonding. Base-stacking and hydrophobic interactions may also contribute to duplex stability.

In this application, a statement that one sequence is the same as or is complementary to another sequence encompasses situations where both of the sequences are completely the same or complementary to one another, and situations where only a portion of one of the sequences is the same as, or is complementary to, a portion or the entire other sequence. Further, a statement that one sequence is complementary to another sequence encompasses situations in which the two sequences have mismatches. Despite the mismatches, the two sequences should selectively hybridize to one another under appropriate conditions.

The term “primer” or “oligonucleotide primer” as used herein, refers to an oligonucleotide from which a primer extension product can be synthesized under suitable conditions. In some embodiments, such suitable conditions comprise the primer being hybridized to a complementary nucleic acid and incubated in the presence of, for example, nucleotides, a polymerization-inducing agent, such as a DNA or RNA polymerase, at suitable temperature, pH, metal concentration, salt concentration, etc. In some embodiments, primers are 5 to 100 nucleotides long. In some embodiments, primers are 8 to 75, 10 to 60, 10 to 50, 10 to 40, or 10 to 35 nucleotides long.

The term “allele-specific primer,” as used herein, refers to a primer that is complementary to a region of a locus that comprises at least one nucleotide position that is different between at least two alleles of the locus. The term “locus-specific primer,” as used herein, refers to a primer that is complementary to a region of a locus that is the same for more than one allele of the locus. In some embodiments, a locus-specific primer is complementary to a region of a locus that is the same for all alleles of the locus.

The term “ligation” as used herein refers to the covalent joining of two polynucleotide ends. In some embodiments, ligation involves the covalent joining of a 3′ end of a first polynucleotide to a 5′ end of a second polynucleotide. In some embodiments, ligation results in a phosphodiester bond (including, for example, a phosphodiester bond in which one or both of the oxygen atoms in the ester bonds are replaced with sulfur (phosphorothioester, such as R₁—O₃P—S—R₂), selenium (phosphoroselenoester, such as R₁—O₃P—Se—R₂), or tellurium (phosphorotelluroester, such as R₁—O₃P—Te—R₂)) being formed between the polynucleotide ends. In some embodiments, ligation may be mediated by any enzyme, chemical, or process that results in a covalent joining of the polynucleotide ends.

The term “chemical ligation” as used herein refers to the covalent joining of two polynucleotide ends that occurs in the absence of an enzyme. In some embodiments, chemical ligation occurs between a polynucleotide comprising a 3′ nucleophile and a polynucleotide comprising a 5′ leaving group.

The term “analyte” or “target analyte” as used herein refers to a substance to be detected using one or more proximity detection probes. Such substances include, but are not limited to, peptides, proteins, carbohydrates, polysaccharides, hormones, small molecules, moieties on the surface of cells, moieties on the surface of microorganisms, and any other substance for which a covalent analyte binding moiety and/or a non-covalent analyte binding moiety can be developed. In some embodiments, an analyte is a protein. In some embodiments, the protein may be a G-protein coupled receptor. In some embodiments, the protein is selected from an enzyme and a receptor. In some embodiments, the enzyme may be a cytochrome P450 or a kinase. An analyte is not a nucleic acid.

The term “sample” as used herein refers to any sample that comprises a nucleic acid suspected of containing a polymorphism, and any sample suspected of containing at least one target analyte. Exemplary samples include, but are not limited to, prokaryotic cells, eukaryotic cells, tissue samples, viral particles, bacteriophage, infectious particles, pathogens, fungi, food samples, bodily fluids (including, but not limited to, mucus, blood, plasma, serum, urine, saliva, and semen), water samples, and filtrates from, e.g., water and air. Exemplary samples also include, but are not limited to, lysates of prokaryotic cells, eukaryotic cells, tissue samples, viral particles, bacteriophage, infectious particles, pathogens, fungi, food samples, and bodily fluids.

A “proximity detection probe” as used herein, is a probe that comprises at least one analyte binding moiety connected, either covalently or noncovalently, to at least one oligonucleotide moiety. An analyte binding moiety may be a covalent analyte binding moiety, or a non-covalent analyte binding moiety. In some embodiments, an analyte binding moiety comprises a first member of a binding pair and the oligonucleotide moiety comprises a second member of a binding pair, wherein the first member of the binding pair and the second member of the binding pair are capable of stably associating under the conditions used for proximity detection probe binding and ligation. In some embodiments, one skilled in the art can select an appropriate binding pair. In some embodiments, a proximity detection probe comprises one or more linkers connecting an analyte binding moiety to an oligonucleotide moiety. In some embodiments, one skilled in the art can select an appropriate linker.

A “covalent analyte binding moiety” as used herein, refers to a moiety that binds specifically and non-covalently to an analyte and subsequently reacts to form a covalent bond to the analyte at or near the site of the non-covalent binding. The non-covalent binding may occur during enzyme catalysis, simple binding to an enzyme active site, or simple binding to any binding site on the analyte. In some embodiments, a covalent analyte binding moiety preferentially attaches to an active analyte, such as an active enzyme or a receptor that is able to bind ligand. In some embodiments, at least the portion of the covalent analyte binding moiety that covalently attaches to an analyte is a small molecule. In some embodiments, a covalent analyte binding moiety comprises a member of a binding pair.

A “non-covalent analyte binding moiety” as used herein, refers to a moiety that specifically and non-covalently binds to a target analyte, but does not covalently attach to the analyte. Such a moiety may bind to the analyte, with a dissociation constant of about 10⁻³ M to about 10⁻¹⁵ M. Exemplary moieties that may be used as non-covalent analyte binding moieties include, but are not limited to, monoclonal antibodies and fragments thereof that are capable of binding an analyte, polyclonal antibodies and fragments thereof that are capable of binding an analyte, proteins, peptides, lectins, nucleic acids, aptamers, carbohydrates, soluble cell surface receptors, small molecules, and any other binding moieties that are specific for a target analyte. In some embodiments, a non-covalent analyte binding moiety comprises a member of a binding pair.

The term “proximity ligation assay” or “PLA” as used herein refers to an assay that involves contacting an analyte with at least two proximity detection probes, wherein a first probe comprises a first analyte binding moiety and a first oligonucleotide moiety and a second probe comprises an analyte binding moiety and a second oligonucleotide moiety. The oligonucleotide moiety of each probe may be the same or different. In some embodiments, the oligonucleotide moiety of each probe in a set of proximity detection probes comprises a different sequence. In some embodiments, the analyte is contacted with a set of proximity detection probes. In some embodiments, a set of proximity detection probes comprises 2, 3, 4, 5, or more than 5 proximity detection probes. In some embodiments, a set of proximity detection probes is a pair of proximity detection probes, or a “proximity detection probe pair.” In some embodiments, the first analyte binding moiety and the second analyte binding moiety in a set of proximity detection probes are capable of interacting with the same analyte. In some embodiments, the first analyte binding moiety and the second analyte binding moiety in a set of proximity detection probes are capable of interacting with different analytes.

In some embodiments, after contacting one or more analytes with at least two proximity detection probes, the oligonucleotide moieties of at least two of the proximity detection probes are capable of interacting with one another. In some embodiments, such interaction may be mediated by one or more additional oligonucleotides. In some embodiments, at least a portion of each of the oligonucleotide moieties of the proximity detection probes hybridizes to another oligonucleotide. For example, in some embodiments, at least one additional oligonucleotide is added (referred to herein as a “splint oligonucleotide”), which mediates the interaction between at least two proximity detection probes by hybridizing to at least a portion of the oligonucleotide moiety of each of the proximity detection probes.

In some embodiments, the oligonucleotide moieties of at least two of the proximity detection probes are capable of undergoing chemical ligation. In some embodiments, the ligatable ends of each of the oligonucleotide moieties are brought together by a splint oligonucleotide that is capable of hybridizing to at least a portion of the oligonucleotide moiety of each proximity detection probe.

Following chemical ligation of the oligonucleotide moieties of at least two proximity detection probes, the ligated oligonucleotide moieties may be detected by any method known in the art. In some such embodiments, the ligated oligonucleotide moieties are referred to as a “target nucleic acid.” Exemplary methods of detecting the ligated oligonucleotide moieties (or “target nucleic acid”) include, but are not limited to, direct detection, real-time PCR (including, but not limited to, 5′-nuclease real-time PCR), rolling circle amplification, combinations of ligation and PCR, and amplification followed by a detection step (such as a second amplification, direct detection, ligation, etc.). Nonlimiting exemplary methods of detecting nucleic acids are described herein.

Exemplary proximity detection assays are described, e.g., in U.S. Pat. No. 6,511,809 B2; U.S. Patent Publication No. US 2002/0064779; PCT Publication No. WO 2005/123963; U.S. Provisional Application No. 61/362,616, filed Jul. 8, 2010; and Gustafsdottir et al., Clin. Chem. 52: 1152-1160 (2006).

The term “quantitative nucleic acid detection assay” as used herein refers to an assay that is capable of quantitating the amount of a particular nucleic acid sequence in a sample. Nonlimiting exemplary quantitative nucleic acid detection assays are described herein.

As used herein, the term “detector probe” refers to a molecule used in an amplification reaction that facilitates detection of the amplification product. Exemplary amplification reactions include, but are not limited to, quantitative PCR, real-time PCR, and end-point analysis amplification reactions. In some embodiments, such detector probes can be used to monitor the amplification of a target nucleic acid and/or control nucleic acid. In some embodiments, detector probes present in an amplification reaction are suitable for monitoring the amount of amplicon(s) produced as a function of time.

In some embodiments, a detector probe is “sequence-based,” meaning that it detects an amplification product in a sequence-specific manner. As a non-limiting example, a sequence-based detector probe may comprise an oligonucleotide that is capable of hybridizing to a specific amplification product. In some embodiments, a detector probe is “sequence-independent,” meaning that it detects an amplification product regardless of the sequence of the amplification product.

Detector probes may be “detectably different,” which means that they are distinguishable from one another by at least one detection method. Detectably different detector probes include, but are not limited to, detector probes that emit light of different wavelengths, detector probes that absorb light of different wavelengths, detector probes that scatter light of different wavelengths, detector probes that have different fluorescent decay lifetimes, detector probes that have different spectral signatures, detector probes that have different radioactive decay properties, detector probes of different charge, and detector probes of different size. In some embodiments, a detector probe emits a fluorescent signal.

The term “detectable label,” as used herein, refers to a moiety that is directly or indirectly detectable. In some embodiments, and as a non-limiting example, a detectable label may be directly detectable, e.g., due to its spectral properties. In some embodiments, and as a non-limiting example, a detectable label may be indirectly detectable, e.g., due to its enzymatic activity, wherein the enzymatic activity produces a directly detectable signal. Such detectable labels include, but are not limited to, radiolabels; pigments, dyes, and other chromogens; spin labels; fluorescent labels (i.e., fluorophores such as coumarins, cyanines, benzofurans, quinolines, quinazolinones, indoles, benzazoles, borapolyazaindacenes, and xanthenes, including fluoresceins, rhodamines, and rhodols); chemiluminescent substances, wherein the detectable signal is generated by chemical modification of substance; metal-containing substances; enzymes, wherein the enzyme activity generates a signal (such as, for example, by forming a detectable product from a substrate; haptens that can bind selectively to another molecule (such as, for example, an antigen that binds to an antibody; or biotin, which binds to avidin and streptavidin). Many detectable labels are known in the art, some of which are described, e.g., in Richard P. Haugland, Molecular Probes Handbook of Fluorescent Probes and Research Products (9^{th} edition, CD-ROM, September 2002), supra. In some embodiments, the detectable label comprises a chromophore, fluorophore, fluorescent protein, phosphorescent dye, tandem dye, particle, hapten, enzyme, or radioisotope. In some embodiments, the fluorophore is a xanthene, coumarin, cyanine, pyrene, oxazine, borapolyazaindacene, or carbopyranine. In some embodiments, the enzyme is horseradish peroxidase, alkaline phosphatase, beta-galactosidase, or beta-lactamase. In some embodiments, the particle is a semiconductor nanocrystal.

“Endpoint polymerase chain reaction” or “endpoint PCR” is a polymerase chain reaction method in which the presence or quantity of nucleic acid target sequence is detected after the PCR reaction is complete, and not while the reaction is ongoing.

“Real-time polymerase chain reaction” or “real-time PCR” is a polymerase chain reaction method in which the presence or quantity of nucleic acid target sequence is detected while the reaction is ongoing. In some embodiments, the signal emitted by one or more detector probes present in a reaction composition is monitored at multiple time points during the PCR as an indicator of synthesis of a primer extension product. In some embodiments, fluorescence emitted at multiple time points during the PCR is monitored as an indicator of synthesis of a primer extension product. In some embodiments, the signal is detected during each cycle of PCR.

A “multiplex amplification reaction” is an amplification reaction in which two or more target nucleic acid sequences and/or control nucleic acid sequences are amplified in the same reaction. A “multiplex polymerase chain reaction” or “multiplex PCR” is a polymerase chain reaction method in which two or more target nucleic acid sequences and/or control nucleic acid sequences are amplified in the same reaction.

A “singleplex amplification reaction” is an amplification reaction in which only one target nucleic acid sequence or control nucleic acid sequence is amplified in the reaction. A “singleplex polymerase chain reaction” or “singleplex PCR” is a polymerase chain reaction method in which only one target nucleic acid sequence or control nucleic acid sequence is amplified in the reaction.

“Threshold cycle” or “C_{T}” is defined as the cycle number at which the observed signal from a quantitative nucleic acid detection assay exceeds a fixed threshold. In some embodiments, the fixed threshold is set as the amount of signal observed in a reaction lacking a target nucleic acid sequence or control nucleic acid sequence. In some embodiments, the fixed threshold is set at a level above the background noise signal. For example, in some embodiments, the fixed threshold is set at a value corresponding to 3 or more times the combination of the root mean squared of the background noise signal and the background noise signal. In some embodiments, the observed signal is from a detector probe. In some embodiments, the observed signal is from a fluorescent label.

The term “solid support” as used herein refers to any solid substance that can be mixed or contacted with a liquid and then separated from the liquid. Separation from the liquid may comprise, in some embodiments, centrifugation, use of a magnet, filtration, settling, pipetting, etc. Nonlimiting exemplary solid supports include microparticles (such as polymer beads, metal particles, magnetic beads, etc.), microtiter plates (such as 96-well plates, 384-well plates, 1536-well plates, etc.), and microarray chips. In some embodiments, a solid support comprises a coating that facilitates binding of, for example, a covalent analyte binding moiety and/or a non-covalent analyte binding moiety and/or an oligonucleotide moiety. In some embodiments, the coating comprises a first member of a binding pair. In some such embodiments, a covalent analyte binding moiety and/or a non-covalent analyte binding moiety and/or an oligonucleotide moiety comprises a second member of the binding pair.

The term “solid support particle,” as used herein, refers to solid support microparticles. Nonlimiting exemplary solid support particles include polymer beads, metal particles, glass beads, and magnetic beads.

### Exemplary Reagents

Exemplary Oligonucleotide Moieties for Chemical Ligation

In some embodiments, an oligonucleotide or oligonucleotide moiety may comprise one or more of ribonucleotides, deoxyribonucleotides, analogs of ribonucleotides, and/or analogs deoxyribonucleotides. Exemplary analogs of ribonucleotides and analogs of deoxyribonucleotides include, but are not limited to, analogs that comprise one or more modifications to the nucleotide sugar, phosphate, and/or base moiety. Exemplary oligonucleotide analogs include, but are not limited to, LNA (see, e.g., U.S. Pat. No. 6,316,198), PNA (see, e.g., U.S. Pat. No. 6,451,968), and any other nucleotide analogs discussed herein or known in the art (see, e.g., Loakes, Nucleic Acids Res. 2001 Jun. 15; 29(12):2437-47, and Karkare et al., Appl Microbiol Biotechnol. 2006 August; 71(5):575-86. Epub 2006 May 9). In some embodiments, an oligonucleotide or oligonucleotide moiety comprises at least one deoxy-uracil (dU) nucleotide in place of at least one deoxy-thymine (dT) nucleotide.

One skilled in the art can select appropriate sequences and lengths for the oligonucleotides and oligonucleotide moieties used in the present methods, according to the intended use. A discussion of nonlimiting exemplary methods of selecting oligonucleotide moieties for proximity detection probes and/or splint oligonucleotides can be found, e.g., in U.S. Pat. No. 6,511,809 B2 and PCT Publication No. WO 2005/123963.

In some embodiments, an oligonucleotide moiety for chemical ligation comprises a 3′ nucleophile. Nonlimiting exemplary 3′ nucleophiles include phosphorothioate, phosphoroselenoate, phosphorotelluroate, thiol, thiocarboxylate, dithiocarboxylate, amino, hydrazine, hydroxylamine, selenol, selenocarboxylate, and diselenocarboxylate. In some embodiments, a 3′ nucleophile is selected from phosphorothioate, phosphoroselenoate, and phosphorotelluroate. In some embodiments, a 3′ nucleophile is selected from phosphorothioate and phosphoroselenoate. In some embodiments, a 3′ nucleophile is phosphorothioate.

In some embodiments, the 3′ terminal nucleotide of an oligonucleotide moiety for chemical ligation comprises the structure:

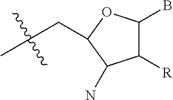

wherein:

B is a nucleobase selected from adenine, guanine, cytosine, uracil, thymine, and derivatives of adenine, guanine, cytosine, uracil, and thymine;

R is selected from H and —OH; and

N is selected from phosphorothioate, phosphoroselenoate, phosphorotelluroate, thiol, thiocarboxylate, dithiocarboxylate, amino, hydrazine, hydroxylamine, selenol, selenocarboxylate, and diselenocarboxylate.

In some embodiments, an oligonucleotide moiety for chemical ligation comprises a 5′ leaving group. Nonlimiting exemplary 5′ leaving groups include, but are not limited to, I, Br, Cl, mesylate, tosylate, brosylate, para-nitrobenzenesulfonate, trifluoromethanesulfonate, trifluoroethanesulfonate, nonafluorobutanesulfonate, trifluoroacetate, a sulfonium cation, and a quaternary ammonium cation. In some embodiments, a 5′ leaving group is selected from I, Br, and Cl.

In some embodiments, the 5′ terminal nucleotide of an oligonucleotide moiety for chemical ligation comprises the structure:

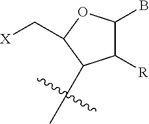

wherein:

B is a nucleobase selected from adenine, guanine, cytosine, uracil, thymine, and derivatives of adenine, guanine, cytosine, uracil, and thymine;

R is selected from H and —OH; and

X is selected from I, Br, Cl, mesylate, tosylate, brosylate, para-nitrobenzenesulfonate, trifluoromethanesulfonate, trifluoroethanesulfonate, nonafluorobutanesulfonate, trifluoroacetate, a sulfonium cation, and a quaternary ammonium cation.

In some embodiments, a first oligonucleotide moiety comprises a 3′ nucleophile and a second oligonucleotide moiety comprises a 5′ leaving group. In some embodiments, a first oligonucleotide moiety comprises a 3′ nucleophile selected from phosphorothioate, phosphoroselenoate, and phosphorotelluroate, and a second oligonucleotide moiety comprises a 5′ leaving group selected from I, Br, and tosylate.

Briefly, in some embodiments, chemical ligation proceeds as follows. A first oligonucleotide moiety comprising a 3′ nucleophile and a second oligonucleotide moiety comprising a 5′ leaving group are brought together such that the 5′ leaving group is adjacent to the 3′ nucleophile, for example, by hybridizing to a template (or splint) oligonucleotide. The 3′ nucleophile then displaces the 5′ leaving group, for example, in an S_{N}2 reaction. As a nonlimiting example, in which the 3′ nucleophile is a phosphorothioate and the 5′ leaving group is an iodo leaving group, and the nucleotides shown are deoxyribonucleotides:

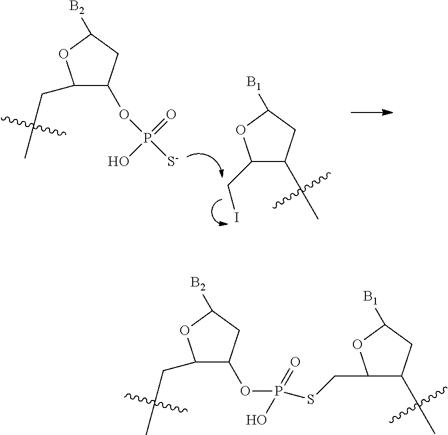

Nonlimiting exemplary chemical ligation and oligonucleotides for chemical ligation are described, e.g., in U.S. Publication Nos. US 2004/0259102; US 2005/0208503; US 2006/0160125; US/2008/0124810; US 2010/0267585; US 2010/0092988; Silverman et al., Chem. Rev. 106: 3775-3789 (2006); Xu et al., Nature Biotech. 19: 148 (2001); and Sando et al., J. Am. Chem. Soc. 126:1081 (2004).

Exemplary Locus-Specific Primers and Allele-Specific Primers

Locus-specific primers are primers that are complementary a region of a locus that is the same for at least two alleles of the locus. In some embodiments, a locus-specific primer is complementary to a region of a locus that is the same for all alleles of the locus. An allele-specific primer is a primer that is complementary to a region of a locus that comprises at least one nucleotide position that is different between at least two alleles of the locus. In some embodiments, an allele-specific primer is a primer that is complementary to a region of a locus that comprises at least one nucleotide position that is different between at least three or at least four alleles of the locus.

In some embodiments, a locus-specific primer is complementary to a region of a locus that is adjacent to the region of the locus to which an allele specific primer is complementary. In some embodiments, an allele-specific primer is complementary to a region of a locus that comprises a single nucleotide polymorphism. In some such embodiments, the 3′ terminal nucleotide of the allele-specific primer hybridizes to the nucleotide of the single nucleotide polymorphism. In some embodiments, the 5′ terminal nucleotide of the allele-specific primer hybridizes to the nucleotide of the single nucleotide polymorphism.

As a nonlimiting example, for a region of a hypothetical locus comprising a single nucleotide polymorphism (SNP), wherein the SNP may be A or G, there are two alleles (the ellipse represents the continuation of the sequence):

Allele 1:
(SEQ ID NO: 18)
5′- . . . AGGTCTGGATGGTCAAAGTTCG . . . -3′
Allele 1:
(SEQ ID NO: 19)
5′- . . . AGGTCTGGATAGTCAAAGTTCG . . . -3′

Some nonlimiting exemplary locus-specific primers may have the sequence (the ellipse represents optional additional complementary and/or noncomplementary sequence):

Locus-specific primer 1:
(SEQ ID NO: 20)
5′- . . . CGAACTTTGAC-3′
Locus-specific primer 2:
(SEQ ID NO: 21)
5′- . . . CGAACTTTGA-3′
Locus-specific primer 3:
(SEQ ID NO: 22)
5′- . . . AGGTCTGGAT-3′
Locus-specific primer 4:
(SEQ ID NO: 23)
5′- . . . AGGTCTGGA-3′

Some nonlimiting exemplary allele-specific primers may have the sequence (the ellipse represents optional additional complementary and/or noncomplementary sequence):

Allele-specific primer 1:
(SEQ ID NO: 24)
5′- . . . AGGTCTGGATG-3′
Allele-specific primer 2:
(SEQ ID NO: 25)
5′- . . . AGGTCTGGATA-3′
Allele-specific primer 3:
(SEQ ID NO: 26)
5′- . . . AGGTCTGGATGG-3′
Allele-specific primer 4:
(SEQ ID NO: 27)
5′- . . . AGGTCTGGATAG-3′
Allele-specific primer 5:
(SEQ ID NO: 28)
5′- . . . CGAACTTTGACC-3′
Allele-specific primer 6:
(SEQ ID NO: 29)
5′- . . . CGAACTTTGACT-3′
Allele-specific primer 7:
(SEQ ID NO: 30)
5′-GGTCAAAGTTCG . . . -3′
Allele-specific primer 8:
(SEQ ID NO: 31)
5′-TGGTCAAAGTTCG . . . -3′

In some embodiments, an allele-specific primer comprises a 3′ nucleophile. In some such embodiments, a locus-specific primer comprises a 5′ leaving group. Further, in some such embodiments, the allele-specific primer hybridizes to a region of the locus that is adjacent to the region of the locus to which the locus-specific hybridizes, such that the 3′ end of the allele-specific primer is adjacent to the 5′ end of the locus-specific primer. In some such embodiments, the allele-specific primer and the locus-specific primer are capable of undergoing chemical ligation when both are hybridizes to the locus.

In some embodiments, an allele-specific primer comprises a 5′ leaving group. In some such embodiments, a locus-specific primer comprises a 3′ nucleophile. Further, in some such embodiments, the allele-specific primer hybridizes to a region of the locus that is adjacent to the region of the locus to which the locus-specific hybridizes, such that the 5′ end of the allele-specific primer is adjacent to the 3′ end of the locus-specific primer. In some such embodiments, the allele-specific primer and the locus-specific primer are capable of undergoing chemical ligation when both are hybridizes to the locus.

Each primer may be any length. For example, a primer may comprises, in some embodiments, 6 to 200 nucleotides (nt), 8 to 200 nt, 10 to 200 nt, 10 to 100 nt, 10 to 75 nt, 10 to 50 nt, etc. In some embodiments, a primer comprises at least 6, at least 7, at least 8, at least 9, at least 10, at least 11, or at least 12 nucleotides. Further, In some embodiments, a primer comprises a first region that is complementary to a region of the locus and a second region that is not complementary to a region of the locus. The second region, in some embodiments, may comprise a sequence that hybridizes to a primer sequence, for example, for amplifying chemically ligated allele- and locus-specific primers.

Exemplary Proximity Detection Probes

A proximity detection probe comprises at least one analyte binding moiety and at least one oligonucleotide moiety. An analyte binding moiety may be a covalent analyte binding moiety or a non-covalent analyte binding moiety. A non-covalent analyte binding moiety is capable of binding to a selected analyte. A covalent analyte binding moiety is capable of covalently attaching to a selected analyte. In some embodiments, a proximity detection probe comprises one analyte binding moiety and one oligonucleotide moiety. In some embodiments, a proximity detection probe comprises more than one analyte binding moiety. In some embodiments, a proximity detection probe comprises more than one oligonucleotide moiety. Nonlimiting exemplary multivalent proximity probes are described, e.g., in U.S. Patent Publication No. US 2005/0003361 A1 to Fredriksson.

In some embodiments, the oligonucleotide moiety of a proximity detection probe comprises one or more of ribonucleotides, deoxyribonucleotides, analogs of ribonucleotides, and/or analogs of deoxyribonucleotides. Exemplary analogs of ribonucleotides and analogs of deoxyribonucleotides include, but are not limited to, analogs that comprise one or more modifications to the nucleotide sugar, phosphate, and/or base moiety. Exemplary oligonucleotide analogs include, but are not limited to, LNA (see, e.g., U.S. Pat. No. 6,316,198), PNA (see, e.g., U.S. Pat. No. 6,451,968), and any other nucleotide analogs known in the art. See, e.g., Loakes, Nucleic Acids Res. 2001 Jun. 15; 29(12):2437-47; and Karkare et al., Appl. Microbiol. Biotechnol. 2006 August; 71(5):575-86. Epub 2006 May 9.

In some embodiments, the oligonucleotide moiety of a proximity detection probe comprises a 3′ nucleophile. Nonlimiting exemplary 3′ nucleophiles include phosphorothioate, phosphoroselenoate, phosphorotelluroate, thiol, thiocarboxylate, dithiocarboxylate, amino, hydrazine, hydroxylamine, selenol, selenocarboxylate, and diselenocarboxylate. In some embodiments, the oligonucleotide moiety of a proximity detection probe comprises a 5′ leaving group. Nonlimiting exemplary 5′ leaving groups include I, Br, Cl, mesylate, tosylate, brosylate, para-nitrobenzenesulfonate, trifluoromethanesulfonate, trifluoroethanesulfonate, nonafluorobutanesulfonate, trifluoroacetate, a sulfonium cation, and a quaternary ammonium cation.

In some embodiments, the oligonucleotide moiety of the proximity detection probe comprises at least 10, at least 15, at least 20, at least 25, at least 30, at least 35, at least 40, at least 50, at least 60, at least 75, or at least 100 nucleotides. In some embodiments, the oligonucleotide moiety of the proximity detection probe comprises 10 to 1000 nucleotides, 10 to 500 nucleotides, 10 to 200, or 10 to 100 nucleotides.

The oligonucleotide moiety and the analyte binding moiety of the proximity detection probe may be covalently or non-covalently associated with one another. Many ways of covalently and non-covalently associating an analyte binding moiety and an oligonucleotide moiety are known in the art.

In some embodiments, the oligonucleotide moiety comprises a first member of a binding pair and the analyte binding moiety comprises a second member of a binding pair, wherein the first member of the binding pair and the second member of the binding pair are capable of stably associating under the conditions used for proximity detection probe binding and/or oligonucleotide hybridization and/or chemical ligation. In some embodiments, the binding pair stably associates through a non-covalent interaction. In some embodiments, the binding pair stably associates through a covalent interaction. In some embodiments, the binding pair need not stably associate during detection of the ligated oligonucleotide moieties. In some embodiments, the binding pair need not stably associate during the initial binding of the analyte binding moiety to the analyte.

Exemplary binding pairs include, but are not limited to, antibody/antigen, biotin and biotin derivatives/avidin and avidin derivatives, biotin and biotin derivatives/streptavidin and streptavidin derivatives, hybridizing nucleic acids, receptor/ligand, folic acid/folate binding protein, vitamin B 12/intrinsic factor, protein A/Fc, and protein G/Fc, metal/chelator, and moieties capable of undergoing a click reaction, etc.

In some embodiments, the analyte binding moiety is associated with an oligonucleotide moiety through a biotin or biotin derivative and a streptavidin or streptavidin derivative. In some embodiments, the analyte binding moiety comprises biotin or a biotin derivative. In some such embodiments, the oligonucleotide moiety comprises streptavidin or a streptavidin derivative. In some embodiments, the analyte binding moiety comprises streptavidin or a streptavidin derivative. In some such embodiments, the oligonucleotide moiety comprises biotin or a biotin derivative. Nonlimiting exemplary biotin derivatives and streptavidin derivatives are described, e.g., in U.S. Publication No. US 2008/0255004. In some embodiments, streptavidin or a streptavidin derivative may be attached to an oligonucleotide moiety by the use of a sulfo-SMCC reagent (see, e.g., Pierce Catalog #22322). In some embodiments, biotin or a biotin derivative may be attached to an oligonucleotide moiety, for example, by a method described in Misiura et al., Nucl. Acids Res., 18: 4345-4354 (1990); Alves et al., Tetrahedron Letters, 30: 3089-3092 (1989); Pon, R.T., Tetrahedron Letters, 32: 1715-1718 (1991); or U.S. Pat. No. 5,567,811. In some embodiments, the analyte binding moiety may be attached to oligonucleotide moiety using hydrazone chemistry, as exemplified by use of S-HyNic and sulfo-S-4FB (Solulink™ Antibody-Oligonucleotide All-in-One Conjugation Kit, Catalog #A-9202-001).

In some embodiments, an analyte binding moiety comprises a moiety capable of undergoing a click reaction. In some such embodiments, an oligonucleotide moiety comprises a complementary moiety capable of undergoing a click reaction. A complementary moiety capable of undergoing a click reaction refers to a second moiety that is capable of undergoing a click reaction with a first moiety. Nonlimiting complementary moieties capable of undergoing a click reaction include azido moieties/ethynyl moieties, azido moieties/phosphine moieties, azido moieties/dibenzocyclooctyne (DIBO) and DIBO-like moieties, and other click-based chemistries. Nonlimiting exemplary moieties capable of undergoing a click reaction are described, e.g., in U.S. Pat. No. 7,375,234; PCT Publication No. WO 01/68565; and PCT Publication No. WO 2009/067663.

In some embodiments, the analyte binding moiety and the oligonucleotide moiety of the proximity detection probe are covalently associated. The non-covalent analyte binding moiety or the covalent analyte binding moiety and the oligonucleotide moiety of the proximity detection probe may be covalently associated following a click reaction as discussed above, or may be covalently associated through other methods. Certain methods of forming covalent bonds between various molecules are known in the art. For example, nonlimiting exemplary methods of making proximity detection probes are described, e.g., in Gullberg et. al., Proc. Natl. Acad. Sci. 101(22): 8420-8424 (2004).

In some embodiments, the 3′ end or the 5′ end of the oligonucleotide moiety is associated with the analyte binding moiety. In some embodiments, the oligonucleotide moiety is associated with the analyte binding moiety at a location other than the 3′ end or the 5′ end of the oligonucleotide moiety, for example, through one or more nucleotides or modified nucleotides in the oligonucleotide sequence.

In some embodiments, two or more proximity detection probes are combined to form a proximity detection probe set. Each proximity detection probe set comprises at least a first proximity detection probe that comprises a first analyte binding moiety and a first oligonucleotide moiety, and a second proximity detection probe that comprises a second analyte binding moiety and a second oligonucleotide moiety. A proximity detection probe set that comprises a first proximity detection probe and a second proximity detection probe may be referred to as a proximity detection probe pair. The first analyte binding moiety and the second analyte binding moiety in a proximity detection probe set may interact with the same analyte, or may interact with different analytes. In some embodiments, when the first analyte binding moiety interacts with a first analyte, and the second analyte binding moiety interacts with a second analyte, the first and second analyte are capable of associating with one another. In some embodiments, such a proximity detection probe set may, for example, be used to detect the association of the first and second analytes.

In some embodiments, a proximity detection probe is capable of binding to more than one analyte. In some embodiments, when a proximity detection probe comprises a covalent analyte binding moiety, the covalent analyte binding moiety may be capable of covalently attaching to more than one analyte. For example, in some embodiments, a covalent analyte binding moiety is capable of covalently attaching to a particular class or subclass of analytes. Nonlimiting exemplary classes or subclasses of analytes include metalloproteases, cysteine proteases, ubiquitin-specific proteases, cysteine cathepsins, esterases, kinases, histone deacetylases, serine reductases, oxidoreductases, ATPases, and GTPases. Nonlimiting exemplary covalent analyte binding moieties are described, e.g., in Bachovchin et al., Nat. Biotech. 27: 387-394 (2009); Cravatt et al., Ann. Rev. Biochem. 77: 383-414 (2008); Fonovie et al., Curr. Pharmac. Des. 13: 253-261 (2007); Kato et al., Nat. Chem. Biol. 1: 33-38 (2005); Patricelli et al., Biochem. 46: 350-358 (2007); Paulick et al., Curr. Opin. Genet. Dev. 18: 97-106 (2008); Saghatelian et al., PNAS 101: 10000-10005 (2004); Salisbury et al., J. Am. Chem. Soc. 130: 2184-2194 (2008); Salisbury et al. PNAS 104: 1171-1176 (2007); Wright et al., Chem. & Biol. 14: 1043-1051 (2007); Wright et al., JACS 131: 10692-10700 (2009); U.S. Pat. No. 6,872,574 B2; and U.S. Publication Nos. US 2009/0252677 A1 and US 2008/0176841 A1.

In some embodiments, a non-covalent analyte binding moiety of a proximity detection probe is capable of binding to more than one analyte. In some such embodiments, a non-covalent analyte binding moiety is capable of binding to a particular motif or epitope that is found in multiple analytes, such as when the non-covalent analyte binding moiety is an antibody or antibody fragment.

Exemplary Detector Probes

In some embodiments, detection of a chemically ligated oligonucleotide comprises amplification. In some embodiments, a detector probe is used in an amplification reaction to facilitate detection of the amplification product. Nonlimiting exemplary detector probes include, but are not limited to, probes used in a 5′-nuclease assay (for example, TaqMan® probes, described, e.g., in U.S. Pat. No. 5,538,848); stem-loop molecular beacons (see, e.g., U.S. Pat. Nos. 6,103,476 and 5,925,517 and Tyagi and Kramer, 1996, Nature Biotechnology 14:303-308); stemless or linear beacons (see, e.g., WO 99/21881), PNA Molecular Beacons™ (see, e.g., U.S. Pat. Nos. 6,355,421 and 6,593,091); linear PNA beacons (see, e.g., Kubista et al., 2001, SPIE 4264:53-58); non-FRET probes (see, e.g., U.S. Pat. No. 6,150,097); Sunrise®/Amplifluor® probes (U.S. Pat. No. 6,548,250); stem-loop and duplex Scorpion® probes (Solinas et al., 2001, Nucleic Acids Research 29:E96 and U.S. Pat. No. 6,589,743); bulge loop probes (U.S. Pat. No. 6,590,091); pseudo knot probes (U.S. Pat. No. 6,589,250), cyclicons (U.S. Pat. No. 6,383,752); MGB Eclipse™ probe (Epoch Biosciences); hairpin probes (U.S. Pat. No. 6,596,490); peptide nucleic acid (PNA) light-up probes; self-assembled nanoparticle probes; and ferrocene-modified probes. Nonlimiting exemplary detector probes are described, for example, in U.S. Pat. No. 6,485,901; Mhlanga et al., 2001, Methods 25:463-471; Whitcombe et al., 1999, Nature Biotechnology 17:804-807; Isacsson et al., 2000, Molecular Cell Probes 14:321-328; Svanvik et al., 2000, Anal Biochem. 281:26-35; Wolffs et al., 2001, Biotechniques 766:769-771; Tsourkas et al., 2002, Nuc. Acids Res. 30:4208-4215; Riccelli et al., 2002, Nuc. Acids Res. 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc. 14:11155-11161.

In some embodiments, detector probes comprise quenchers. Exemplary quenchers include, but are not limited to, black hole quenchers (Biosearch), Iowa Black (IDT), QSY quencher (Molecular Probes), and Dabsyl and Dabcel sulfonate/carboxylate Quenchers (Epoch). In some embodiments, detector probes comprise two probes, wherein, for example, one probe comprises a fluorescent moiety and another probe comprises a quencher, wherein hybridization of the two probes together on a target quenches the signal, or wherein hybridization of the two probes on a target alters the signal via a change in fluorescence. Nonlimiting exemplary detector probes comprising two probes are described, e.g., in U.S. Patent Publication No. US 2006/0014191 to Lao et al. Exemplary detector probes also include, but are not limited to, sulfonate derivatives of fluorescein dyes with SO₃ instead of the carboxylate group, phosphoramidite forms of fluorescein, and phosphoramidite forms of CY 5 (commercially available, e.g., from Amersham).

In some embodiments, detector probes comprise intercalating labels. Exemplary intercalating labels include, but are not limited to, ethidium bromide, SYBR® Green I (Molecular Probes), and PicoGreen® (Molecular Probes), which allow visualization in real-time, or at an end point, of an amplification product in the absence of a nucleic acid probe. In some embodiments, a detector probe comprising an intercalating label is a sequence-independent detector probe. In some embodiments, real-time visualization can comprise a sequence-independent intercalating detector probe and a sequence-based detector probe.

In some embodiments, a detector probe is at least partially quenched when not hybridized to a complementary sequence in the amplification reaction, and is at least partially unquenched when hybridized to a complementary sequence in the amplification reaction. In some embodiments, detector probes further comprise various modifications, such as, for example, a minor groove binder (see, e.g., U.S. Pat. No. 6,486,308) to further provide desirable thermodynamic characteristics. In some embodiments, detector probes can correspond to identifying portions or identifying portion complements, also referred to as zip-codes. Identifying portions are described, e.g., in U.S. Pat. No. 6,309,829 (referred to as a “tag segment” therein); U.S. Pat. No. 6,451,525 (referred to as a “tag segment” therein); U.S. Pat. No. 6,309,829 (referred to as a “tag segment” therein); U.S. Pat. No. 5,981,176 (referred to as “grid oligonucleotides” therein); U.S. Pat. No. 5,935,793 (referred to as “identifier tags” therein); and PCT Publication No. WO 01/92579 (referred to as “addressable support-specific sequences” therein).

### Exemplary Methods

Methods provided herein may be carried out in any order of the recited events that is logically possible, as well as the recited order of events.

Standard techniques may be used for recombinant DNA, oligonucleotide synthesis, and tissue culture. Enzymatic reactions and purification techniques may be performed according to manufacturer's specifications and/or as commonly accomplished in the art and/or as described herein. The foregoing techniques and procedures may be generally performed according to conventional methods known in the art and as described in various general and more specific references, including but not limited to, those that are cited and discussed throughout the present specification. See, e.g., Sambrook et al. Molecular Cloning: A Laboratory Manual (2d ed., Cold Spring Harbor Laboratory Press, Cold Spring Harbor, N.Y. (1989)); Lehninger, Biochemistry (Worth Publishers, Inc.); Methods in Enzymology (S. Colowick and N. Kaplan Eds., Academic Press, Inc.); Oligonucleotide Synthesis (N. Gait, ed., 1984); A Practical Guide to Molecular Cloning (2.sup.nd Ed., Wily Press, 1988). Unless specific definitions are provided, the nomenclatures utilized in connection with, and the laboratory procedures and techniques of, biology, biochemistry, analytical chemistry, and synthetic organic chemistry described herein are those known and used in the art.

Exemplary Detection of Polymorphisms

Methods of detecting single nucleotide polymorphisms (SNPs) are provided. In some embodiments, a method of detecting a SNP comprises hybridizing a locus-specific primer to a region of a locus, and hybridizing an allele-specific primer to a region of a locus, such that the locus-specific primer and the allele-specific primer are capable of undergoing chemical ligation. In some embodiments, the locus-specific primer comprises a 5′ leaving group and the allele-specific primer comprises a 3′-nucleophile. In some embodiments, the locus-specific primer comprises a 3′-nucleophile and the allele-specific primer comprises a 5′ leaving group. In some such embodiments, the locus-specific primer and the allele-specific primer hybridize to their respective regions of the locus such that the 5′ leaving group is adjacent to the 3′-nucleophile such that chemical ligation can occur.

In some embodiments, the allele-specific primer comprises a nucleotide at the 3′ end that is specific for on allele of the SNP. In some embodiments, the allele-specific primer comprises a nucleotide at the 5′ end that is specific for one allele of the SNP. In some embodiments, the nucleotide in the allele-specific primer that is specific for one allele of the SNP is located within 2, 3, 4, 5, or 6 bases of the 3′ or 5′ end of the allele-specific primer.

In some embodiments, hybridization and chemical ligation of the locus-specific primer and the allele-specific primer occurs on purified genetic material. In some embodiments, hybridization and chemical ligation of the locus-specific primer and the allele-specific primer occurs on an amplified copy of a region of the locus.

In some embodiments, a polymorphism detection assay comprises at least one locus-specific primer and at least one allele-specific primer. In some embodiments, a polymorphism detection assay comprises one locus-specific primer and at least two allele-specific primers. In some such embodiments, each of the allele-specific primers is specific for a different nucleotide present at a SNP location. That is, as a nonlimiting example, if a SNP can be A or G, one allele-specific primer is specific for the “A” allele, and one allele-specific primer is specific for the “G” allele. Similarly, as a further nonlimiting example, if a SNP can be A, G, or T, one allele-specific primer is specific for the “A” allele, one allele-specific primer is specific for the “G” allele, and one allele-specific primer is specific for the “T” allele, and so on. In some embodiments, a polymorphism detection assay detects more than one polymorphism simultaneously. In some such embodiments, the assay comprises at least two locus-specific primers and at least two allele-specific primers, wherein a first locus-specific primer and at least one first allele-specific primer hybridize to a first locus, a second locus-specific primer and at least one second allele-specific primer hybridize to a second locus, etc.

Following chemical ligation of a locus-specific primer and an allele-specific primer, the chemically ligated oligonucleotide may be detected. In some embodiments, each allele-specific primer and/or each locus-specific primer is detectable different. In some embodiments, each allele-specific primer and/or each locus-specific primer comprises a different detectable label. In some embodiments, each allele-specific primer and/or each locus-specific primer comprises a portion that is not complementary to the locus, but has a sequence that is different from at least some of the other primers in the assay, such that each chemically ligated oligonucleotide can be separately detected. In some embodiments, each allele-specific primer and/or each locus-specific primer comprises a member of a binding pair. In some embodiments, each allele-specific primer and/or each locus-specific primer comprises a member of a different binding pair, such that each chemically ligated oligonucleotide can be separately detected.

In some embodiments, an allele-specific primer comprises a detectable label and a locus-specific primer comprises a member of a binding pair. In some embodiments, each of two or more allele-specific primers comprises a detectably different label and a locus-specific primer comprises a member of a binding pair. In some embodiments, a locus-specific primer comprises a detectable label and an allele-specific primer comprises a member of a binding pair. In some embodiments, following chemical ligation, the chemically ligated oligonucleotide is bound to a solid phase comprising the second member of the binding pair. In some such embodiments, association of a particular detectable label with the solid phase indicates that a particular allele is present. In some embodiments, rather than binding to a solid phase after chemical ligation an oligonucleotide moiety (e.g., a primer) is bound to a solid phase before chemical ligation takes place. Such binding to a solid phase may be through a binding pair, or through other means, such as, for example, covalent attachment.

In some embodiments, the relative abundance of two or more different alleles can be determined using chemical ligation. For example, in some embodiments, if two alleles are present in equal amounts, such as in a heterozygous individual, the relative abundance of the detectable labels associated with each allele-specific primer will be approximately equal. In a homozygous individual, in some embodiments, the relative abundance of the detectable label associated with one allele-specific primer will be much greater than the detectable label associated with another allele-specific primer.

Exemplary Proximity Ligation Assays

Methods of detecting an analyte in a sample are provided. In some embodiments, the methods facilitate detection of active analyte in a sample. The methods comprise forming a complex comprising an analyte, a first proximity detection probe, and a second proximity detection probe, wherein the first proximity detection probe comprises a first analyte binding moiety and a first oligonucleotide moiety and the second proximity detection probe comprises a second analyte binding moiety and a second oligonucleotide moiety. In some embodiments, the first oligonucleotide moiety comprises a 3′-phosphorothioate or a 3′-phosphoroselenoate and the second oligonucleotide moiety comprises a 5′ leaving group. In some embodiments, the first oligonucleotide moiety and the second oligonucleotide moiety are contacted with a splint oligonucleotide that hybridizes with at least a portion of the first oligonucleotide moiety and at least a portion of the second oligonucleotide moiety. In some embodiments, the first oligonucleotide and the second oligonucleotide undergo chemical ligation in the presence of the splint oligonucleotide. In some embodiments, detecting the interaction of the first oligonucleotide moiety and the second oligonucleotide moiety comprises amplification. In some embodiments, detecting the interaction comprises quantitative PCR.

Formation of a complex comprising an analyte, a first proximity detection probe, and a second proximity detection probe can be accomplished using many different reagents and through a series of steps carried out in many different orders. That is, in some embodiments, the complex is formed by (a) contacting a target analyte (TA) with a first analyte binding moiety (ABM1) that comprises a first member of a binding pair; (b) contacting the TA-ABM1 complex with a first oligonucleotide moiety (O1) that comprises a second member of the binding pair; (c) contacting the TA-ABM1-O1 complex with a proximity detection probe comprising a second analyte binding moiety and a second oligonucleotide moiety (ABM2-O2), thus forming complex O2-NABM-TA-CABM-O1, which comprises a target analyte, a first proximity detection probe, and a second proximity detection probe.

In some embodiments, steps (b) and (c) are carried out in reverse order (in which case, an O2-ABM2-TA-ABM1 complex is formed after the second step) or are carried out simultaneously. In some embodiments, step (c) is carried out before steps (a) and (b), in which case, an O2-ABM2-TA complex is formed before step (a) is carried out (which forms an O2-ABM2-TA-ABM1 complex). In some embodiments, all of the steps are carried out simultaneously.

In some embodiments, the complex is formed by (a) contacting a target analyte (TA) with a first analyte binding moiety (ABM1) that comprises a first member of a first binding pair; (b) contacting the TA-ABM1 complex with a second analyte binding moiety (ABM2) that comprises a first member of a second binding pair; (c) contacting the ABM2-TA-ABM1 complex with a first oligonucleotide moiety (O1) that comprises a second member of the first binding pair; (d) contacting the ABM2-TA-ABM1-O1 complex with a second oligonucleotide moiety (O2) that comprises a second member of the second binding pair, thus forming complex O2-ABM2-TA-ABM1-O1, which comprises an analyte, a first proximity detection probe, and a second proximity detection probe. In some embodiments, steps (a) and (b) are carried out in reverse order (in which case, an ABM2-TA complex is formed after the first step) or simultaneously. In some embodiments, steps (c) and (d) are carried out in reverse order (in which case, an O2-ABM2-TA-ABM1 is formed after the third step) or are carried out simultaneously. In some embodiments, the order of steps is (a) and (c), in that order or simultaneously (forming a TA-ABM1-O1 complex), then (b) and (d), in that order or simultaneously. In some embodiments, the order of steps is (b) and (d), in that order or simultaneously (forming an O2-ABM2-TA complex), then (a) and (c), in that order or simultaneously. In some embodiments, all of the steps are carried out simultaneously.

In some embodiments, a complex is formed by (a) contacting a target analyte (TA) with a proximity detection probe that comprises a first analyte binding moiety and a first oligonucleotide moiety (AMB1-O1); and (b) contacting the TA-ABM1-O1 complex with a proximity detection probe comprising a second analyte binding moiety and a second oligonucleotide moiety (ABM2-O2), thus forming complex O2-ABM2-TA-ABM1-O1, which comprises an analyte, a first proximity detection probe, and a second proximity detection probe. In some embodiments, steps (a) and (b) are carried out in reverse order (thus forming an O2-ABM1-TA complex after the first step), or are carried out simultaneously.

In some embodiments, one or more of the steps described above for forming a complex comprising an analyte, a first proximity detection probe, and a second proximity detection probe is carried out in a lysate of a biological sample. In some embodiments, the lysate is a prokaryotic cell lysate, a eukaryotic cell lysate, a viral lysate, a bacteriophage lysate, or a tissue lysate. In some embodiments, one or more of the steps described above for forming the complex are carried out on whole cells. In some such embodiments, the target analyte is located on the surface of a cell, and one or more of the steps described above for forming the complex are carried out without lysing the cells. In some embodiments, all of the steps described above for forming the complex are carried out without lysing the cells. In some embodiments, detecting the interaction between the first oligonucleotide moiety and the second oligonucleotide moiety is carried out without lysing the cells.

In some embodiments, the target analyte is located within cells, and at least one of the steps described above for forming a complex comprising an analyte, a first proximity detection probe, and a second proximity detection probe is carried out without lysing the cells. In some embodiments, a TA-ABM1 complex is formed without lysing cells. In some such embodiments, the first analyte binding moiety is a covalent analyte binding moiety. In some embodiments, following formation of the TA-ABM1 complex, the cells are lysed before the remaining components are bound to the complex. In some such embodiments, the first member of a binding pair comprised in the first analyte binding moiety is a moiety capable of undergoing a click reaction. In some such embodiments, an oligonucleotide moiety comprises a second member of the binding pair, such as a complementary click moiety. In some embodiments, the first member of a binding pair comprised in the first analyte binding moiety is a biotin or biotin derivative. In some such embodiments, an oligonucleotide moiety comprises a second member of the binding pair, such as a streptavidin or streptavidin derivative.

In some embodiments, the target analyte is located within a multicellular organism and at least one of the steps described above for forming a complex comprising an analyte, a first proximity detection probe, and a second proximity detection probe is carried out in the living organism. In some embodiments, the organism is administered, or contacted with, a first analyte binding moiety comprising a first member of a binding pair. In some such embodiments, the first analyte binding moiety is a covalent analyte binding moiety. In some embodiments, following formation of a TA-ABM1 complex, a sample is removed from the organism and the remaining components are bound to the complex. In some embodiments, the remaining components are bound to the complex following lysis of the sample removed from the organism. In some embodiments, the remaining components are bound to the complex without lysing the sample removed from the organism.

In some embodiments, more than one proximity detection probe sets are bound to their respective target analytes in the same mixture. That is, in some embodiments, the steps described above for forming a complex are carried out to form more than one different complex simultaneously. Thus, for example, in some embodiments, a first analyte binding moiety comprising a first member of a first binding set and a second analyte binding moiety comprising a first member of a second binding set are incubated with the same sample to form a TA1-ABM1 complex and a TA2-ABM2 complex. The TA1-ABM1 complex and TA2-ABM3 complex are then contacted with a first oligonucleotide moiety comprising a second member of the first binding set and a second oligonucleotide moiety comprising a second member of the second binding set, to form a TA1-ABM1-O1 complex and a TA2-ABM2-O2 complex. The TA1-ABM1-O1 complex and TA2-ABM2-O2 complex are then contacted with a first proximity detection probe comprising a third analyte binding moiety and a third oligonucleotide moiety, and a second proximity detection probe comprising a fourth analyte binding moiety and a fourth oligonucleotide moiety, to form an O3-ABM3-TA1-ABM1-O1 complex and an O4-ABM4-TA2-ABM2-O2 complex. As discussed above, many different ways of forming the final complexes are contemplated, and every complex in a mixture need not have been formed in the same way.

In some embodiments, one or more of the steps discussed above for forming a complex comprising an analyte, a first proximity detection probe, and a second proximity detection probe, are carried out on a solid support. In some embodiments, a analyte binding moiety and/or an oligonucleotide moiety is bound to a solid support. An analyte binding moiety and/or an oligonucleotide moiety may be bound to a solid support noncovalently or covalently. In some embodiments, an analyte binding moiety and/or an oligonucleotide moiety is reversibly bound to a solid support. In some embodiments, an analyte binding moiety and/or an oligonucleotide moiety is bound to a solid support using a binding pair. In some embodiments, when an analyte binding moiety and/or an oligonucleotide moiety is bound to a solid support, one or more steps in forming a complex comprising an analyte, a first proximity detection probe, and a second proximity detection probe is followed and/or preceded by at least one wash step. In some embodiments, each step is followed and/or preceded by a wash step. In some embodiments, not all of the steps are followed and/or preceded by a wash step.

In some embodiments, at least a portion of detecting the chemical ligation between a first oligonucleotide moiety and a second oligonucleotide moiety occurs on a solid phase. In some embodiments, detecting the chemical ligation between a first oligonucleotide moiety and a second oligonucleotide moiety occurs in solution.

In some embodiments, at least one splint oligonucleotide is added to the sample before, at the same time as, or after addition of at least one proximity detection probe. In some embodiments, the splint oligonucleotide is capable of hybridizing to at least a portion of the oligonucleotide moiety of the first proximity detection probe, and is also capable of hybridizing to at least a portion of the oligonucleotide moiety of the second proximity detection probe. In some embodiments, the hybridized region between the splint oligonucleotide(s) and an oligonucleotide moiety of a proximity detection probe comprises at least 5 base pairs, at least 10 base pairs, at least 15 base pairs, at least 20 base pairs, at least 25 base pairs, at least 30 base pairs, at least 40 base pairs, at least 50 base pairs, at least 75 base pairs, or at least 100 base pairs. In some embodiments, a splint oligonucleotide is symmetric, e.g., it hybridizes to an equal number of bases of each oligonucleotide moiety. In some embodiments, a splint oligonucleotide is asymmetric, e.g., it hybridizes to a greater number of bases of one oligonucleotide moiety than of the other oligonucleotide moiety. Nonlimiting exemplary splint oligonucleotides are described, e.g., in PCT Publication No. WO 2005/123963.

In some embodiments, a splint oligonucleotide hybridizes to the first and second oligonucleotide moieties in such a way that the 3′ end of one of the oligonucleotide moieties is adjacent to the 5′ end of the other oligonucleotide moieties. In some embodiments, the 3′ and 5′ ends of the oligonucleotide moieties of the proximity detection probe pair are capable of undergoing chemical ligation.

After the splint oligonucleotide is added to the sample, in some embodiments, the chemical ligation reaction is incubated for at least 2 minutes, at least 5 minutes, at least 10 minutes, at least 15 minutes, at least 30 minutes, or at least 1 hour. In some embodiments, the ligation reaction is incubated for 20 to 200 minutes, for 20 to 100 minutes, for 20 to 90 minutes, or for 30 to 60 minutes. In some embodiments, the ligation reaction is incubated at a temperature between 10° C. to 65° C., between 15° C. and 65° C., between 15° C. and 65° C., between 20° C. and 65° C., or between 25° C. and 65° C. In some embodiments, the ligation reaction is incubated at a temperature greater than 25° C. In some embodiments, the ligation reaction is incubated at about 50° C.

In some embodiments, at least one splint oligonucleotide is added to the sample before, at the same time as, or after at least one proximity detection probe is added to the sample. For example, in some embodiments, where at least one splint oligonucleotide is added after the proximity detection probe set addition and incubation, the sample is further incubated at a temperature and for a time sufficient to allow hybridization of the at least one splint oligonucleotide to at least one proximity detection probe set. In some embodiments, the sample is incubated at a temperature and for a time sufficient to allow chemical ligation between a first oligonucleotide moiety and a second oligonucleotide moiety. In some embodiments, one skilled in the art can select an appropriate time and temperature for such hybridization and/or chemical ligation. In some embodiments, conditions include temperatures between 0° C. to 75° C. In some embodiments, the incubation is carried out at between 0° C. and 90° C., between 4° C. and 90° C., between 10° C. and 75° C., or between 25° C. and 60° C. In some embodiments, the incubation is carried out for at least 5 minutes, at least 10 minutes, at least 30 minutes, at least an hour, or at least 2 hours.

In some embodiments, after a complex comprising an analyte, a first proximity detection probe, a second proximity detection probe, and a splint oligonucleotide is formed, the sample is treated with at least one protease. In some embodiments, after chemical ligation of the first oligonucleotide moiety and the second oligonucleotide moiety, the sample is treated with at least one protease. In some embodiments, after addition of the at least one protease, the sample is incubated for at least 5 minutes, at least 10 minutes, at least 15 minutes, at least 30 minutes, at least 1 hour, at least 2 hours, or at least 4 hours. In some embodiments, the sample is incubated at at least one temperature between 0° C. to 65° C., between 0° C. and 55° C., between 4° C. and 50° C., between 10° C. and 45° C., or between 15° C. and 40° C. In some embodiments, at least one protease is inactivated after incubation. In some embodiments, at least one protease is heat inactivated, e.g., by incubating the sample for at least 5 minutes at at least 50° C. In some embodiments, the sample is incubated at at least 55° C., at least 60° C., at least 65° C., at least 70° C., or at least 75° C. to heat inactivate the protease. In some embodiments, at least one protease is inactivated, e.g., by addition of at least one chemical. In some embodiments, at least one protease is inactivated by addition of PMSF.

In some embodiments, after inactivation of the at least one protease, the ligated proximity detection probe sets are detected. In some embodiments, one or more proximity detection probe sets are detected using the same detection method. In some embodiments, one or more proximity detection probe sets are detected simultaneously. In some embodiments, detection of the at least one ligated proximity detection probe sets comprises multiplex quantitative PCR. In some embodiments, detection of the at least one ligated proximity detection probe sets comprises singleplex quantitative PCR. In some embodiments, the method does not comprise a nucleic acid purification step prior to detection of the one or more proximity detection probe sets. For example, in some embodiments, a different label is used to detect each different proximity detection probe set.

In some embodiments, the ligated product and the hybridized splint oligonucleotide are subjected to a primer extension reaction as part of, or prior to, the detection method. In some embodiments, the primer extension reaction produces a double-stranded oligonucleotide. In some embodiments, the primer extension reaction comprises at least one oligonucleotide primer complimentary to the ligated product. In some embodiments, the splint oligonucleotide serves as a primer in the primer extension reaction, along with a second oligonucleotide primer. In some embodiments, two oligonucleotide primers other than the splint oligonucleotide are included in the primer extension reaction. In some embodiments, following a primer extension reaction that produces a double-stranded oligonucleotide, a first strand of the double stranded oligonucleotide comprises the ligated oligonucleotide moieties, and the second strand comprises the sequence of the splint oligonucleotide connected to (i) a first sequence that is complementary to at least a portion of the first oligonucleotide moiety, and also connected to (ii) a second sequence that is complementary to at least a portion of the second oligonucleotide moiety.

In some embodiments, when the detection method involves hybridization of one or more oligonucleotides (such as, for example, one or more oligonucleotide primers and/or detector probes comprising oligonucleotides), one skilled in the art can select an appropriate nucleotide sequence such that the oligonucleotide can be used to specifically detect the ligated product. For example, in some embodiments, where the ligated oligonucleotide moieties are subjected to a primer extension reaction, one or more oligonucleotides that hybridize to the primer extension product and not to the oligonucleotide moieties or the splint oligonucleotide can be selected. Such oligonucleotides may be used, in some embodiments, in a direct detection method and/or in a detection method involving an amplification step. In some embodiments, one or more oligonucleotides can be selected to amplify the ligated oligonucleotide moieties such that amplification will only occur if the moieties are ligated together.

Exemplary Normalizer Controls for Proximity Ligation Assays

In some embodiments, the amount of a target analyte may be normalized to at least one normalizer control. Nonlimiting exemplary normalizer controls are described, e.g., herein and in PCT Publication No. WO 2005/123963. In some embodiments, one skilled in the art can select one or more normalizer controls for a particular application.

A normalizer control may be “exogenous” or “endogenous.” In some embodiments, an exogenous normalizer control is added to a sample after the sample is collected. In some embodiments, the sample naturally comprises an amount of the same analyte that is used as an exogenous normalizer control, but the normalizer control is considered to be exogenous because an additional amount of analyte has been added.

In some embodiments, an endogenous normalizer control is already present in a sample at the time the sample is collected for analysis. A normalizer control is referred to as “housekeeping,” in some embodiments, when it is present at a high level in a biological sample without having been added. In some embodiments, a housekeeping normalizer control is present at a high level in more than one different type of biological sample.

In some embodiments, a normalizer control is an endogenous analyte. In some embodiments, a normalizer control is an endogenous protein. In some embodiments, a normalizer control is an endogenous enzyme. In some embodiments, a normalizer controls is an endogenous housekeeping protein. Exemplary endogenous housekeeping protein normalizer controls include, but are not limited to, GAPDH, acidic ribosomal protein, beta-actin, HPRT, beta-glucuronidase, cystatin B, ICAM1, and p53.

In some embodiments, a normalizer control is an exogenous analyte. In some embodiments, a normalizer control is an exogenous protein. In some embodiments, a normalizer control is an exogenous enzyme. Exemplary exogenous protein normalizer controls include, but are not limited to, bacterial proteins, protein tags, viral proteins, intact virions, insect proteins, mammalian proteins not normally expressed in the selected biological sample, and mammalian proteins normally expressed at a low level in the selected biological sample. In some embodiments, a normalizer control is an enzyme. In some embodiments, a normalizer control is the same class or subclass of enzyme as the target analyte. In some embodiments, a normalizer control is a receptor. In some embodiments, a normalizer control is the same class or subclass of receptor as the target analyte.

In some embodiments, a sample comprises at least one normalizer control, at least two normalizer controls, at least three normalizer controls, at least four normalizer controls, or at least five normalizer controls. In some embodiments, a sample comprises at least one endogenous normalizer control and at least one exogenous normalizer control. In some embodiments, all of the normalizer controls in a sample are endogenous. In some embodiments, all of the normalizer controls in a sample are exogenous.

In some embodiments, a normalizer control is detected in the same sample in which a target analyte is detected. In some embodiments, a normalizer control is detected in the same vessel in which a target analyte is detected, using the same or different methods. In some embodiments, the sample is split or divided and a normalizer control and a target analyte are detected in separate vessels, using the same or different methods. In some embodiments, a normalizer control is detected at the same time that a target analyte is detected.

In some embodiments, the amount of a target analyte may be normalized to a normalizer control using the “comparative C_{T} method” or “ΔC_{T} method,” which involves calculating the ΔC_{T}. In some embodiments, the ΔC_{T} is calculated by subtracting the C_{T} of a quantitative nucleic acid detection assay used to detect a normalizer control from the C_{T} of a quantitative nucleic acid detection assay used to detect a target analyte. In some embodiments, the fold difference in the amounts of the normalizer control and target analyte is calculated from the ΔC_{T}. In some embodiments, the fold difference in the amounts of the normalizer control and target analyte is calculated from the ΔC_{T} according to the formula 2^{-ΔCT}.

In some embodiments, the ΔC_{T} is calculated by subtracting the ΔC_{T} of a “calibrator sample” from the ΔC_{T} of a “test sample.” Exemplary calibrator samples include, but are not limited to, a sample prepared from untreated cells and a sample prepared from a particular tissue. Exemplary test samples include, but are not limited to, a sample prepared from treated cells and a sample prepared from a tissue other than the tissue from which a calibrator sample was prepared. In some embodiments, the ΔΔC_{T} is calculated by subtracting the ΔC_{T} of a calibrator sample from the ΔC_{T} of a test sample.

In some embodiments, the fold difference in the amount of target nucleic acid in the calibrator and test samples is calculated from the ΔΔC_{T} according to the formula 2^{-ΔCT}. In some embodiments, the fold difference in the amount of target analyte in the calibrator and test samples is calculated from the ΔΔC_{T} according to the formula 2^{-ΔCT}. Use of the ΔΔC_{T} method is described, e.g., in Applied Biosystems, “Guide to Performing Relative Quantitation of Gene Expression Using Real-Time Quantitative PCR” (2008); and Applied Biosystems, User Bulletin #2: ABI Prism 7700 Sequence Detection System (Dec. 11, 1997 (updated October 2001)).

In some embodiments, the use of a normalizer control may eliminate the need to prepare an external standard curve using an analyte, which may produce a C_{T} value that differs from the C_{T} value observed when there is an identical level of the analyte in a sample. In some embodiments, the use of a normalizer control may control for a variable in a proximity ligation assay. Exemplary variables in proximity ligation assays include, but are not limited to, nucleic acid degradation, analyte degradation, the extent to which analyte activity and/or structure has been maintained, the efficiency with which a proximity detection probe interacts with an analyte, the efficiency of a ligation reaction, and the efficiency of a real-time PCR reaction.

In some embodiments, an analyte normalizer control is detected using a proximity ligation assay. Nonlimiting exemplary proximity ligation assays are described herein. In some embodiments, an analyte normalizer control is detected using the same method (using appropriate proximity detection probes) and in the same vessel as a target analyte. In some embodiments, an analyte normalizer control is detected using the same method (using appropriate proximity detection probes) but in a different vessel as a target analyte.

### Exemplary Detection of Chemically Ligated Oligonucleotides

In some embodiments, multiple chemically ligated oligonucleotides are detected simultaneously in the same vessel. In some embodiments, multiple ligated oligonucleotides are detected simultaneously in a multiplex amplification reaction. In some embodiments, different labels are used to identify the different chemically ligated oligonucleotides. For example, in some embodiments, if three alleles or three target analytes are being detected in a sample, and a single detection reaction is used to detect the chemically ligated oligonucleotides, three different labels may be used to separately identify the different detection reaction products. In some embodiments, such labels may be in the form of detector probes, discussed herein, or any other label known in the art that is suitable for use in the detection methods. One skilled in the art can select an appropriate label or labels, according to some embodiments.

In some embodiments, chemically ligated oligonucleotides are detected using real-time PCR. Exemplary methods of performing real-time PCR include, but are not limited to, 5′ nuclease real-time PCR, and multiplexed versions thereof. Nonlimiting exemplary methods of 5′ nuclease real-time PCR are known in the art and are described, e.g., in Livak, Methods Mol. Biol. 212:129-47 (2003); Lee et al., Biotechniques 27(2):342-9 (1999); Livak, Genet. Anal. 14(5-6):143-9 (1999); Heid et al., Genome Res. 6(10):986-94 (1996); and Lee et al., Nucleic Acids Res. 11; 21(16):3761-6 (1993). Nonlimiting exemplary quantitative PCR is described, e.g., in A-Z Quantitative PCR, Bustin, S., Ed., IUL Biotechnology Series (2004). Nonlimiting exemplary methods of real-time PCR are also described, e.g., in Watson et al., Int J. Toxicol. 2005 May-June; 24(3):139-45; and U.S. Pat. Nos. 6,890,718; 6,773,817; and 6,258,569. In some embodiments, a target nucleic acid is detected using TaqMan One-step qRT-PCR (Applied Biosystems).

In some embodiments, passive reference dyes may be used in quantitative PCR methods. Nonlimiting exemplary passive reference dyes are described, e.g., in U.S. Pat. No. 5,736,333. In some embodiments, external controls may be used in quantitative PCR methods. Nonlimiting exemplary quantitative controls are described, e.g., in U.S. Pat. No. 6,890,718.

In some embodiments, chemically ligated oligonucleotides are amplified in a first “pre-amplification reaction” (described, e.g., in PCT Publication No. WO2004/051218), and then decoded in a second amplification reaction. Certain exemplary such methods are known in the art and are described, e.g., in U.S. Pat. No. 6,605,451; U.S. Pat. No. 7,604,937; and U.S. Pat. No. 7,601,821.

Nonlimiting exemplary methods of detecting chemically ligated oligonucleotides are also described, e.g., in U.S. Pat. No. 6,511,809 B2; U.S. Publication No. US 2002/0064779 A1; and PCT Publication No. WO 2005/123963. Nonlimiting exemplary multiplex detection methods are described, e.g., in U.S. Publication No. US 2006/0216737.

In some embodiments, a detector probe is used to facilitate detection of the ligated oligonucleotides. Nonlimiting exemplary detector probes are discussed herein. In some embodiments, one skilled in the art can select one or more suitable detector probes according to the intended application.

Exemplary Labeling of Solid Support Particles

In some embodiments, methods of labeling solid support particles using chemical ligation are provided. In some embodiments, the method comprises forming a reaction mixture comprising a first oligonucleotide moiety comprising a 5′ leaving group and a detectable label, a second oligonucleotide moiety comprising a 3′ nucleophile and a first member of a binding pair, and a splint oligonucleotide that is capable of hybridizing to at least a portion of the first oligonucleotide moiety and at least a portion of the second oligonucleotide moiety such that the 5′ leaving group and the 3′ nucleophile are adjacent to one another. In some embodiments, the method comprises forming a reaction mixture comprising a first oligonucleotide moiety comprising a 3′ nucleophile and a detectable label, a second oligonucleotide moiety comprising a 5′ leaving group and a first member of a binding pair, and a splint oligonucleotide that is capable of hybridizing to at least a portion of the first oligonucleotide moiety and at least a portion of the second oligonucleotide moiety such that the 5′ leaving group and the 3′ nucleophile are adjacent to one another.

In some embodiments, the method comprises incubating the reaction mixture under conditions allowing chemical ligation of the first oligonucleotide moiety to the second oligonucleotide moiety. In some embodiments, the method comprises including in the reaction mixture a solid support particle comprising a second member of the binding pair before, during, or after chemical ligation of the first oligonucleotide moiety and the second oligonucleotide moiety. In some embodiments, the splint oligonucleotide is removed. Removal of the splint oligonucleotide may be by destabilizing the hybridization of the splint oligonucleotide to the chemically ligated oligonucleotide, e.g., using heat or chemical means.

In some embodiments, the method comprises forming a reaction mixture comprising at least two different oligonucleotide moieties comprising detectably different labels, each comprising a 3′ nucleophile, an oligonucleotide moiety comprising a first member of a binding pair and a 5′ leaving group, and at least two splint oligonucleotides, wherein the first splint oligonucleotide is capable of hybridizing to at least a portion of the first labeled oligonucleotide moiety and at least a portion of the oligonucleotide moiety comprising the first member of the binding pair such that the 5′ leaving group and the 3′ nucleophile are adjacent to one another; and the second splint oligonucleotide is capable of hybridizing to at least a portion of the second labeled oligonucleotide moiety and at least a portion of the oligonucleotide moiety comprising the first member of the binding pair such that the 5′ leaving group and the 3′ nucleophile are adjacent to one another. In some embodiments, the method comprises forming a reaction mixture comprising at least three, at least four, at least five, or at least six labeled oligonucleotide moieties. In some embodiments, the method further comprises forming a reaction mixture comprising at least three, at least four, at least five, or at least six splint oligonucleotides.

In some embodiments, the method comprises incubating the reaction mixture under conditions allowing chemical ligation of pairs of oligonucleotide moieties hybridized to each splint oligonucleotide. In some embodiments, the method comprises including in the reaction mixture a solid support particle comprising a second member of the binding pair before, during, or after chemical ligation of the first oligonucleotide moiety and the second oligonucleotide moiety. Binding of the ligated oligonucleotides to the solid support particles results in labeled solid support particles. In some embodiments, the splint oligonucleotide is removed. Removal of the splint oligonucleotide may be by destabilizing the hybridization of the splint oligonucleotide to the chemically ligated oligonucleotide, e.g., using heat or chemical means.

In some embodiments, a method of labeling solid support particles comprises forming a reaction mixture comprising a first oligonucleotide moiety comprising a 5′ leaving group and a detectable label; a second oligonucleotide moiety comprising a 3′ nucleophile and which is attached, either covalently or non-covalently, to a solid support particle; and a splint oligonucleotide that is capable of hybridizing to at least a portion of the first oligonucleotide moiety and at least a portion of the second oligonucleotide moiety such that the 5′ leaving group and the 3′ nucleophile are adjacent to one another. In some embodiments, the method comprises forming a reaction mixture comprising a first oligonucleotide moiety comprising a 3′ nucleophile and a detectable label; a second oligonucleotide moiety comprising a 5′ leaving group and which is attached, either covalently or non-covalently, to a solid support particle; and a splint oligonucleotide that is capable of hybridizing to at least a portion of the first oligonucleotide moiety and at least a portion of the second oligonucleotide moiety such that the 5′ leaving group and the 3′ nucleophile are adjacent to one another.

In some embodiments, the method comprises incubating the reaction mixture under conditions allowing chemical ligation of the first oligonucleotide moiety to the second oligonucleotide moiety, resulting in labeled solid support particles. In some embodiments, the splint oligonucleotide is removed. Removal of the splint oligonucleotide may be by destabilizing the hybridization of the splint oligonucleotide to the chemically ligated oligonucleotide, e.g., using heat or chemical means.

In some embodiments, the method comprises forming a reaction mixture comprising at least two different oligonucleotide moieties comprising detectably different labels, each comprising a 3′ nucleophile; an oligonucleotide moiety comprising a 5′ leaving group and which is attached, either covalently or non-covalently, to a solid support particle; and at least two splint oligonucleotides, wherein the first splint oligonucleotide is capable of hybridizing to at least a portion of the first labeled oligonucleotide moiety and at least a portion of the oligonucleotide moiety comprising the first member of the binding pair such that the 5′ leaving group and the 3′ nucleophile are adjacent to one another; and the second splint oligonucleotide is capable of hybridizing to at least a portion of the second labeled oligonucleotide moiety and at least a portion of the oligonucleotide moiety comprising the first member of the binding pair such that the 5′ leaving group and the 3′ nucleophile are adjacent to one another. In some embodiments, the method comprises forming a reaction mixture comprising at least three, at least four, at least five, or at least six labeled oligonucleotide moieties. In some embodiments, the method further comprises forming a reaction mixture comprising at least three, at least four, at least five, or at least six splint oligonucleotides.

In some embodiments, the method comprises incubating the reaction mixture under conditions allowing chemical ligation of pairs of oligonucleotide moieties hybridized to each splint oligonucleotide, resulting in labeled solid support particles. In some embodiments, the splint oligonucleotide is removed. Removal of the splint oligonucleotide may be by destabilizing the hybridization of the splint oligonucleotide to the chemically ligated oligonucleotide, e.g., using heat or chemical means.

In some embodiments, the ratios of labeled oligonucleotide moiety (“probe oligo”), oligonucleotide moiety comprising a member of a binding pair and/or which is attached to a solid support particle (“capture oligo”), and splint oligonucleotide are adjusted in order to adjust the intensity and proportion of a label on a solid support particle.

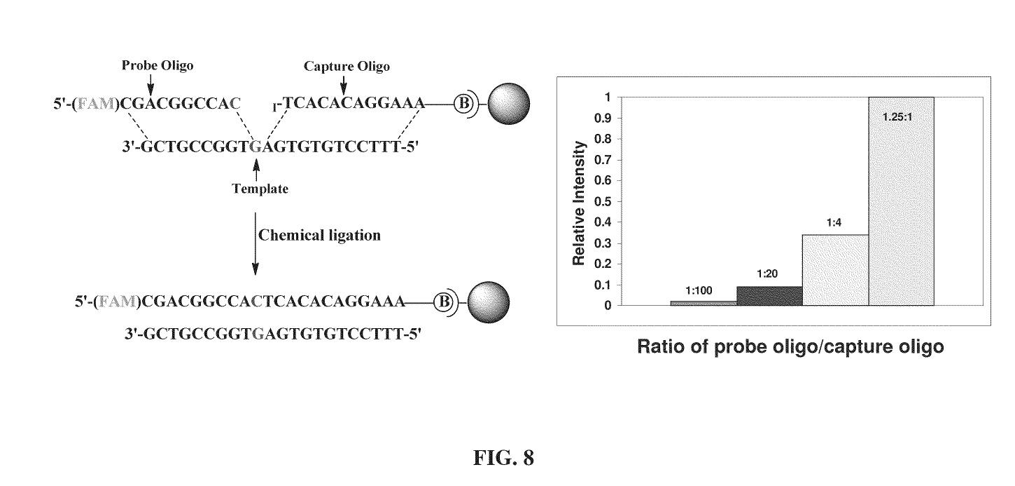

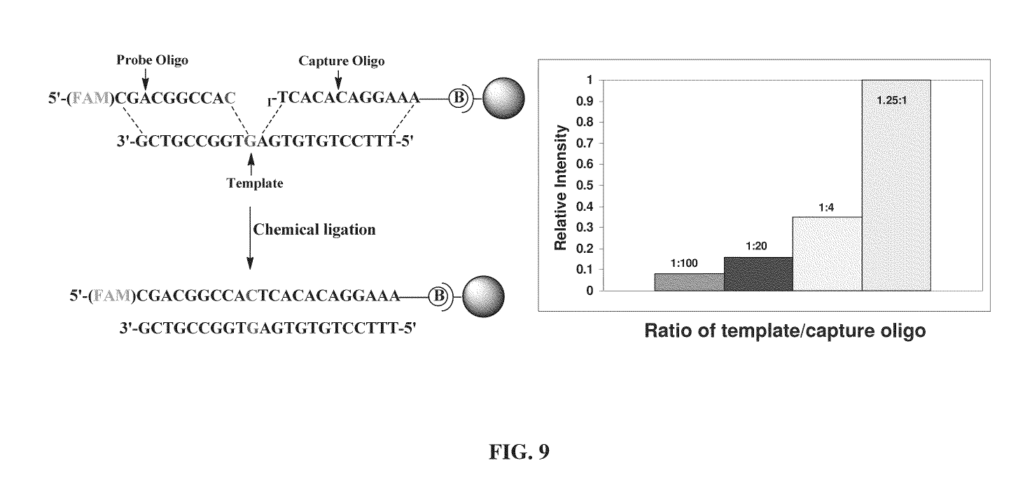

Nonlimiting exemplary labeling of solid support particles are described in Examples 6 and 7 and shown in FIGS. 8 to 11. Briefly, as shown in FIGS. 8 and 9, a solid support particle may be labeled with one color by forming a complex comprising a probe oligo with a 3′ nucleophile, a capture oligo with a 5′ leaving group, and a splint oligo (or “template”). The solid support particle is labeled by chemical ligation of the probe oligo to the capture oligo. Further, the intensity of the labeling can be changed by changing the ratio of probe oligo to capture oligo (see FIG. 8) and/or the ratio of template to capture oligo (see FIG. 9).

In some embodiments, the ratio of probe oligo to capture oligo is between 1:200 and 2:1, between 1:100 and 2:1, between 1:100 and 1.25:1, between 1:20 and 1.25:1, or between 1:4 and 1.25:1. In some embodiments, the ratio of template oligo to capture oligo is kept constant. In some embodiments, the ratio of template oligo to probe oligo is kept constant. In some embodiments, the concentration of template oligo is kept constant. In some embodiments, the ratio of template to capture oligo is between 1:200 and 2:1, between 1:100 and 2:1, between 1:100 and 1.25:1, between 1:20 and 1.25:1, or between 1:4 and 1.25:1. In some embodiments, the ratio of probe oligo to capture oligo is kept constant. In some embodiments, the ratio of probe oligo to template oligo is kept constant. In some embodiments, the concentration of probe oligo is kept constant.

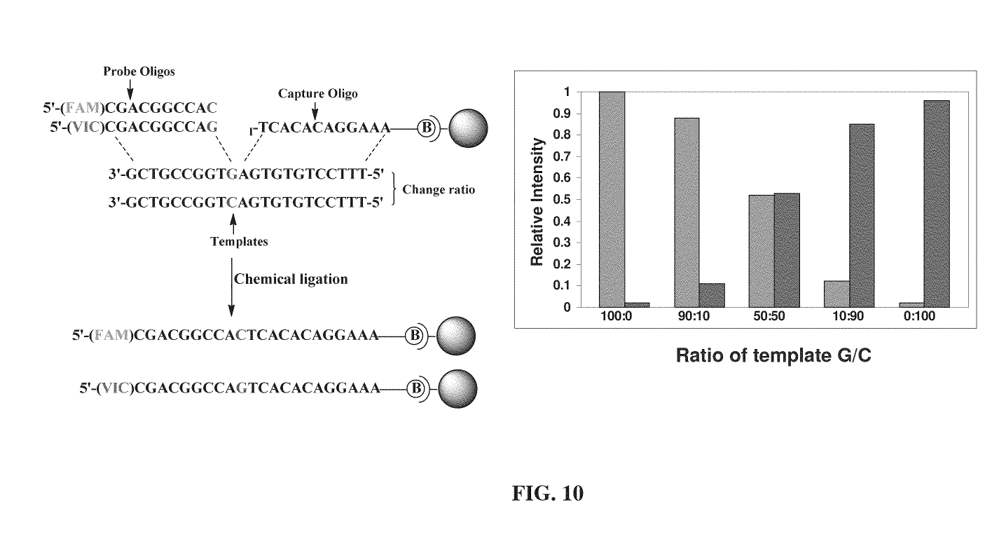

FIG. 10 shows a nonlimiting exemplary method of labeling a solid support with two different detectable labels. In some embodiments, a reaction mixture comprises two probe oligos, wherein each probe oligo comprises a different sequence (in Example 10, the probe oligos differ by one nucleotide at the 3′ terminus) and a different detectable label. In some embodiments, the reaction mixture further comprises a capture oligo and two different templates, one of which hybridizes to one of the probe oligos, and the other of which hybridizes to the other probe oligo. The solid support particle is labeled by chemical ligation of the capture oligos the probe oligos. The ratio and intensity of each of the detectable labels can be changed by changing the ratio of probe oligos to one another, and/or the ratio of probe oligos to capture oligo, and/or the ratio of template oligos to one another, and/or the ratio of template oligos to capture oligo.

In some embodiments, the ratio of probe oligos to one another is between 200:0 and 0:200; or between 100:0 and 0:100. As a nonlimiting example, in some embodiments, in order to obtain, for example, five distinguishable combinations of the two labels, the ratio of the probe oligos to one another may be 100:0, between 90:10 and 70:30, 50:50, between 10:90 and 30:70, and 0:100. In some embodiments, the ratio of templates to one another is between 200:0 and 0:200; or between 100:0 and 0:100. As a nonlimiting example, in some embodiments, in order to obtain, for example, five distinguishable combinations of the two labels, the ratio of the templates to one another may be 100:0, between 90:10 and 70:30, 50:50, between 10:90 and 30:70, and 0:100. In a similar manner, one skilled in the art can vary the ratios of the oligonucleotides and/or oligonucleotide moieties in the reaction composition to obtain changes in label intensities. One skilled in the art can select suitable ratios of probe oligo, capture oligo, and/or template in order to obtain the desired number of distinguishable combinations of detectable labels.

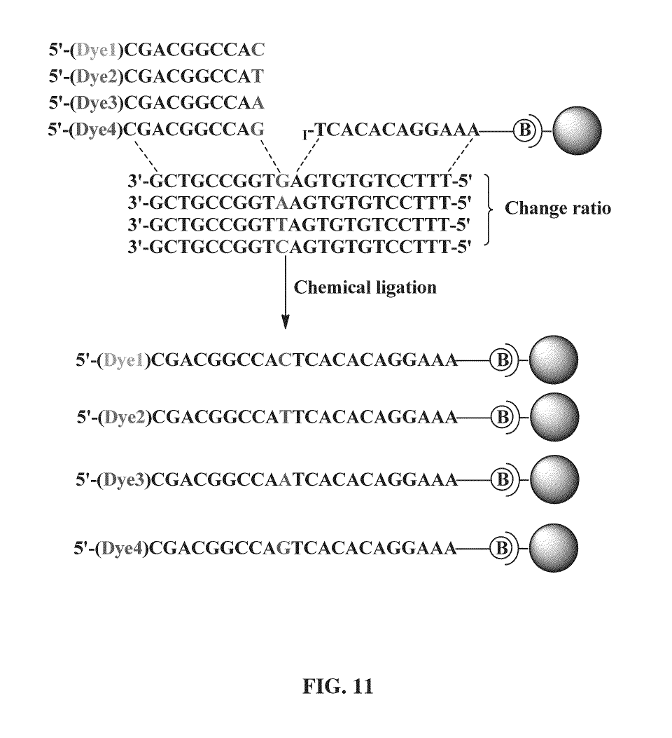

A nonlimiting exemplary method of labeling solid support particles with four different detectable labels, using four different probe oligos and four different templates, is shown in FIG. 11. As discussed above, the ratios of the four probe oligos and/or the four template oligos and/or the capture oligo can be changed by one skilled in the art to obtain the desired number of different label combinations and intensities.

### Exemplary Kits

In some embodiments, kits comprising at least one component for carrying out the methods exemplified herein are provided. In some embodiments, a kit comprises a first allele-specific primer and a locus specific primer. In some such embodiments, the first allele-specific primer comprises a 3′ nucleophile and the locus-specific primer comprises a 5′ leaving group. In some embodiment, the first allele specific primer and the locus-specific primer hybridize to a target nucleic acid such that the 5′ end of the locus-specific primer is adjacent to the 3′ end of the first allele-specific primer. In some embodiments, the kit further comprises a second allele-specific primer that comprises a 3′ nucleophile. In some such embodiments, the second allele-specific primer differs from the first allele-specific primer at least at a nucleotide that hybridizes with a single nucleotide polymorphism.

In some embodiments, a kit comprises a first allele-specific primer and a locus specific primer. In some such embodiments, the first allele-specific primer comprises a 5′ leaving group and the locus-specific primer comprises a 3′ nucleophile. In some embodiment, the first allele specific primer and the locus-specific primer hybridize to a target nucleic acid such that the 3′ end of the locus-specific primer is adjacent to the 5′ end of the first allele-specific primer. In some embodiments, the kit further comprises a second allele-specific primer that comprises a 5′ leaving group. In some such embodiments, the second allele-specific primer differs from the first allele-specific primer at least at a nucleotide that hybridizes with a single nucleotide polymorphism.

In some embodiments, the allele-specific primer and/or the locus-specific primer comprises a detectable label. In some embodiments, the allele-specific primer and/or the locus-specific primer comprises a first member of a binding pair. In some such embodiments, the first member of the binding pair is a biotin or biotin derivative.

In some embodiments, a kit comprises a first proximity detection probe that comprise a first analyte binding moiety and an oligonucleotide moiety, and a second proximity detection probe that comprises a second analyte binding moiety and an oligonucleotide moiety. In some embodiments, a kit comprises a first analyte binding moiety that comprises a first member of a binding pair, and a proximity detection probe that comprises a second analyte binding moiety and an oligonucleotide moiety. In some embodiments, a kit comprises an analyte binding moiety that comprises a first member of a first binding pair, and a second analyte binding moiety that comprises a first member of a second binding pair. In some embodiments, a kit comprises one or more oligonucleotide moieties that comprise second members of binding pairs.

In some embodiments, an oligonucleotide moiety comprises a 3′ nucleophile. In some embodiments, an oligonucleotide moiety comprises a 5′ leaving group. In some embodiments, a kit comprises an oligonucleotide moiety that comprises a 3′ nucleophile and an oligonucleotide moiety that comprises a 5′ leaving group, wherein each oligonucleotide moiety is associate with, or is capable of associating with, an analyte binding moiety. In some embodiments, a kit comprises a splint oligonucleotide. In some such embodiments, the first oligonucleotide moiety and the second oligonucleotide moiety are capable of hybridizing to the splint oligonucleotide such that the 3′ nucleophile and the 5′ leaving group are adjacent to each other and capable of chemically ligating.

In some embodiments, a kit comprises at least one component for detecting a proximity ligation probe set. Exemplary components include, but are not limited to, detector probes, primers, polymerases, and reverse transcriptases.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Rate and Specificity of Chemical Ligation Using DNA and RNA Templates

0.5 μM of a 5′-FAM labeled oligonucleotide moiety with the sequence:

| | |
|---|---|
| 5′-(FAM)CGACGGCCAC-3′ | (SEQ ID NO: 1) |
| or | |
| 5′-(FAM)CGACGGCCAA-3′, | (SEQ ID NO: 2) |

each with a 3′ phosphorothioate, was incubated with 0.5 μM of a 3′-biotinylated oligonucleotide moiety with the sequence:

(SEQ ID NO: 3)
5′-TCACACAGGAAA(PEG)(PEG)(biotin)-3′,

with a 5′ iodo leaving group, and with 0.5 μM of a DNA template (or “splint oligonucleotide”) with the sequence:

| | |
|---|---|
| 5′-TTTCCTGTGTGATTGGCCGTCG-3′ | (SEQ ID NO: 4) |

In TE buffer with 10 mM MgCl₂ at either 37° C. or 50° C. Four separate chemical ligations were carried out in duplicate—one for each of the FAM-labeled oligonucleotides, at each temperature. Ligation yield at 0 hours, 30 minutes, 1 hour, 2 hours, 4 hours, and 8 hours was measured as follows. Ligated product was bound to streptavidin-coated latex beads (6 μm or 10 μm) and free FAM-labeled oligonucleotide moieties washed off with TE buffer at 50° C. FAM bound to the streptavidin-coated beads was measured by flow cytometry using an Attune® Acoustic Focusing Cytometer (Applied Biosystems, Carlsbad, Calif.) and a BD LSR II flow cytometer with 488 nm excitation and a 525/50 emission filter. Blank beads were used as a control to set up the flow cytometer. The PMT voltage setting on the Blue-E channel (with a 525/50 band-pass filter) was adjusted so that the mean fluorescence intensity (MFI) of the blank beads was less than 50. The same instrument settings were used to run the stained beads, collecting 10,000 events for each sample.

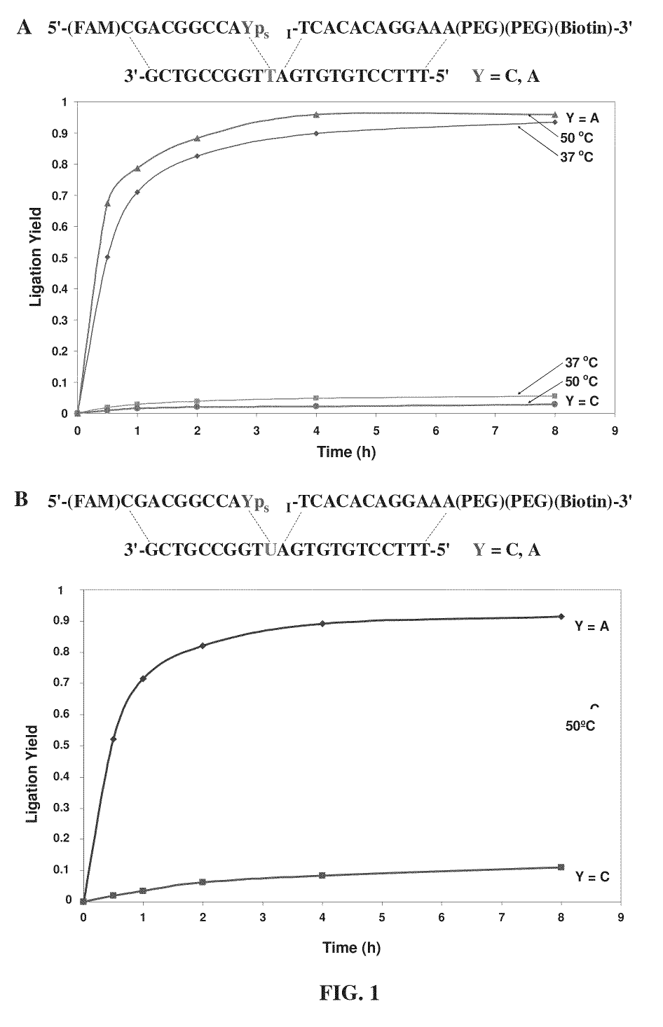

As shown in FIG. 1A, chemical ligation was very specific under both temperatures tested in that experiment. At each temperature, chemical ligation of the FAM-labeled oligonucleotide moiety with an A at the 3′ terminus occurred rapidly in the presence of the template with a T at the position that hybridizes to the 3′-terminal A, while little chemical ligation occurred with the FAM-labeled oligonucleotide moiety with a C at the 3′ terminus. Further, chemical ligation occurred rapidly, reaching 70% of maximum in an hour at 37° C., and almost 80% of maximum in an hour at 50° C.

A similar experiment was carried out using the same FAM-labeled oligonucleotide moieties and biotin-labeled oligonucleotide moiety, but using an RNA template having the sequence:

(SEQ ID NO: 5)
5′-UUUCCUGUGUGAUUGGCCGUCG-3′

In this experiment, the chemical ligations were carried out at 50° C. in TE buffer with 10 mM MgCl₂. Ligation yield was determined as above.

As shown in FIG. 1B, chemical ligation in the presence of an RNA template was very specific in that experiment. Chemical ligation of the FAM-labeled oligonucleotide moiety with an A at the 3′ terminus occurred rapidly in the presence of the RNA template with a T at the position that hybridizes to the 3′-terminal A, while little chemical ligation occurred with the FAM-labeled oligonucleotide moiety with a C at the 3′ terminus. Further, chemical ligation occurred rapidly, reaching 70% of maximum in an hour at 50° C.

### Example 2

### Sensitivity and Specificity of Chemical Ligation Versus Enzymatic Ligation

To compare the sensitivity and specificity of chemical ligation versus enzymatic ligation, experiments were carried out using oligonucleotides of the same sequence, but designed for either chemical or enzymatic ligation. For chemical ligation, 0.5 μM of a FAM-labeled oligonucleotide moiety with the sequence:

| | |
|---|---|
| 5′-(FAM)CGACGGCCAC-3′ | (SEQ ID NO: 1) |

with a 3′ phosphorothioate, was incubated with 0.5 μM of a 3′-biotinylated oligonucleotide moiety with the sequence:

(SEQ ID NO: 3)
5′-TCACACAGGAAA(PEG)(PEG)(biotin)-3′,

with a 5′ iodo leaving group, with or without 0.5 μM of a DNA template with the sequence:

(SEQ ID NO: 4)
5′-TTTCCTGTGTGATTGGCCGTCG-3′

in TE buffer with 10 mM MgCl₂ at 50° C.

For enzymatic ligation, 0.5 μM a FAM-labeled oligonucleotide moiety with the sequence:

(SEQ ID NO: 1)
5′-(FAM)CGACGGCCAC-3′

with a 3′-OH, was incubated with 0.5 μM of a 3′-biotinylated oligonucleotide moiety with the sequence:

(SEQ ID NO: 3)
5′-TCACACAGGAAA(PEG)(PEG)(biotin)-3′,

with a 5′ phosphate, with 0.5 μM of a DNA template with the sequence:

(SEQ ID NO: 4)
5′-TTTCCTGTGTGATTGGCCGTCG-3′

in 50 mM Tris-HCl, 10 mM MgCl₂, 1 mM ATP, 1 mM DTT, and 5% polyethylene glycol-8000, and with or without 40 units of T4 DNA ligase at 37° C.

Following ligation, the ligated products were bound to streptavidin-coated latex beads (6 μm or 10 μm) and free FAM-labeled oligonucleotide moieties were washed off with TE buffer at 50° C. Samples of FAM-bound beads were visualized using a Zeiss Axioskop 2 fluorescence microscope with 40× objective equipped with a Hamamatsu ORCA-ER CCD camera using a 480±10 nm band-pass filter for excitation and a 515±10 nm band-pass filter for emission. The fluorescence intensity of FAM bound to the streptavidin-coated beads was also measured by flow cytometry using an Attune® Acoustic Focusing Cytometer (Applied Biosystems, Carlsbad, Calif.) with 488 nm excitation and a 525/50 emission filter. Blank beads were used as a control to set up the flow cytometer. The PMT voltage setting on the Blue-E channel (with a 525/50 band-pass filter) was adjusted so that the mean fluorescence intensity (MFI) of the blank beads is less than 50. The same instrument settings were used to run the stained beads, collecting 10,000 events for each sample.

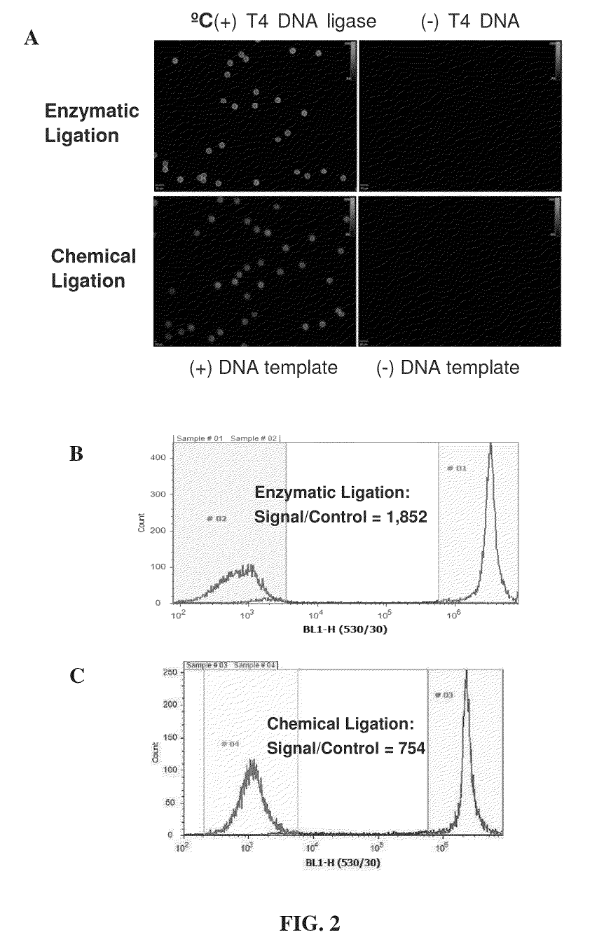

As shown in FIG. 2A, for enzymatic ligation, FAM associated with the streptavidin-coated beads only when T4 DNA ligase was included in the incubation mix. Similarly, for chemical ligation, FAM associated with the streptavidin-coated beads only when DNA template was included in the incubation mix. Panels B and C show the signals obtained for FAM detection on the Attune® Acoustic Focusing Cytometer. The signal to control ratio for the enzymatic ligation was 1,852, while the signal to control ratio for the chemical ligation was 754. These experiments show that both enzymatic and chemical ligation were very sensitive and selective by measuring the fluorescence intensity with a flow cytometer.

### Example 3

### Sequence Selectivity of Chemical Ligation

Chemical ligations between FAM-labeled oligonucleotide moieties with single base mismatches at various positions relative to the DNA template were carried out at 37° C. and 50° C. to determine the sensitivity of chemical ligation to mismatches at the various positions at the two different temperatures. For each chemical ligation, 0.5 μM of the biotin-labeled oligonucleotide moiety with the sequence:

(SEQ ID NO: 3)
5′-TCACACAGGAAA(PEG)(PEG)(biotin)-3′,

with a 5′ iodo leaving group was used, along with 0.5 μM of a DNA template with the sequence:

(SEQ ID NO: 6)
5′-TTTCCTGTGTGACTGGCCGTCG-3′.

The FAM-labeled oligonucleotide moieties tested in this experiment were:

(SEQ ID NO: 7)
5′-(FAM)CGACGGCCAG-3′,
(SEQ ID NO: 1)
5′-(FAM)CGACGGCCAC-3′,
(SEQ ID NO: 8)
5′-(FAM)CGACGGCCTG-3′,
(SEQ ID NO: 9)
5′-(FAM)CGACGGCTAG-3′,
(SEQ ID NO: 10)
5′-(FAM)CGACGGGTAG-3′,
(SEQ ID NO: 11)
5′-(FAM)CGACGCCTAG-3′,
(SEQ ID NO: 12)
5′-(FAM)CGACCGCTAG-3′,

each with a 3′-phosphorothioate. Each chemical ligation was carried out in TE buffer with 10 mM MgCl₂ for 3 hours at 37° C. or at 50° C. Ligated product was bound to streptavidin-coated latex beads (10 μm) and free FAM-labeled oligonucleotide moieties were washed off with TE buffer at 50° C. The fluorescence intensity of FAM bound to the streptavidin-coated beads was measured by flow cytometry using a BD LSR II flow cytometer with 488 nm excitation and a 525/50 emission filter. Blank beads were used as a control to set up the flow cytometer. The PMT voltage setting on the Blue-E channel (with a 525/50 band-pass filter) was adjusted so that the mean fluorescence intensity (MFI) of the blank beads is less than 50. The same instrument settings were used to run the stained beads, collecting 10,000 events for each sample.

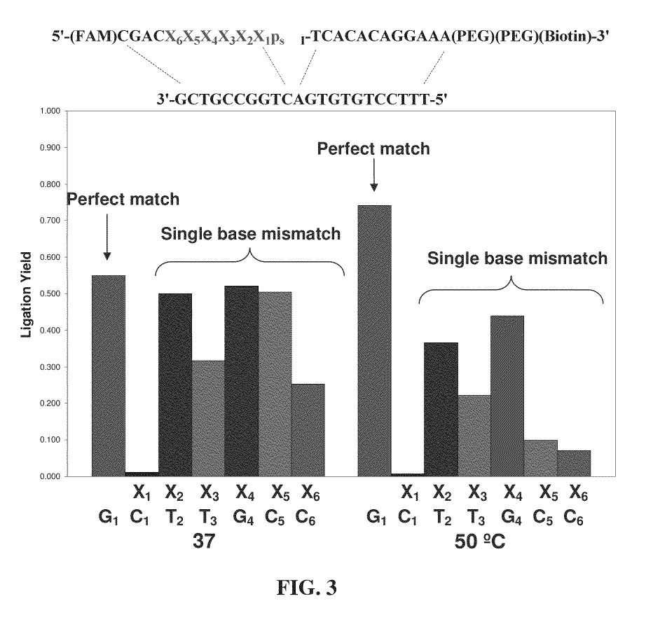

The results of that experiment are shown in FIG. 3. As shown in that figure, the 3′ terminus position (“X₁”) of the FAM-labeled oligonucleotide moiety showed the best selectivity of the six positions tested. Further, the selectivity was greater at 50° C. than at 37° C. for all of the mismatch positions tested. At 50° C., the selectivity of chemical ligation for the perfect match versus a C at the X₁ position was 124-fold.

Next, the selectivity of enzymatic ligation was tested by varying the nucleotide at the 3′ terminus of the FAM-labeled oligonucleotide moiety. In this experiment, Four FAM-labeled oligonucleotide moieties, each with a different 3′ terminal nucleotide, were tested against four DNA templates, each with a different nucleotide at the position that hybridizes to the 3′ terminal nucleotide of the FAM-labeled oligonucleotide moiety. Specifically, the oligonucleotides tested in this experiment were 0.5 μm of FAM-labeled oligonucleotide moieties with the sequences:

(SEQ ID NO: 7)
5′-(FAM)CGACGGCCAG-3′,
(SEQ ID NO: 2)
5′-(FAM)CGACGGCCAA-3′,
(SEQ ID NO: 13)
5′-(FAM)CGACGGCCAT-3′,
(SEQ ID NO: 1)
5′-(FAM)CGACGGCCAC-3′,

each with a 3′-OH; and 0.5 μm of DNA templates with the sequences:

(SEQ ID NO: 6)
5′-TTTCCTGTGTGACTGGCCGTCG-3′,
(SEQ ID NO: 4)
5′-TTTCCTGTGTGATTGGCCGTCG-3′,
(SEQ ID NO: 14)
5′-TTTCCTGTGTGAATGGCCGTCG-3′,
and
(SEQ ID NO: 15)
5′-TTTCCTGTGTGAGTGGCCGTCG-3′.

For each ligation, 0.5 μm of a biotin-labeled oligonucleotide moiety with the sequence:

(SEQ ID NO: 3)
5′-TCACACAGGAAA(PEG)(PEG)(biotin)-3′,

and a 5′ phosphate, was used. Each ligation was carried out in 50 mM Tris-HCl, 10 mM MgCl₂, 1 mM ATP, 1 mM DTT, and 5% polyethylene glycol-8000 and 40 units T4 DNA ligase for 20 minutes at 37° C. FAM-labeled enzymatically ligated products were bound to streptavidin-coated beads and analyzed on an Attune® Acoustic Focusing Cytometer as described above.

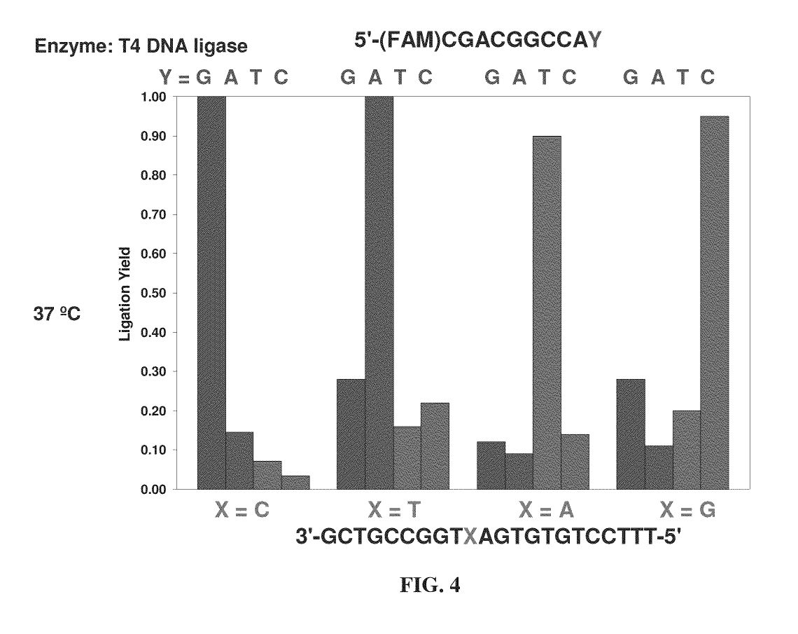

The results of that experiment are shown in FIG. 4. The selectivity of T4 DNA ligase for each combination of 3′-terminal nucleotides and templates ranged from 3-fold (compare Y=G and Y=C for X=G) to 33-fold (compare Y=G and Y=C for X=C), with many combinations showing a selectivity of less than 10-fold.

Table 1 shows the results of the enzymatic ligation experiment.

TABLE 1
Ligation Yield and Selectivity for Enzymatic Ligation

| Base pair | | | Base Pair | Ligation | |
|---|---|---|---|---|---|
| (X-Y) | Ligation yield | Selectivity | (X-Y) | Yield | Selectivity |
| C-G | 1 | — | A-G | 0.12 | 8X |
| C-A | 0.14 | 7X | A-A | 0.09 | 10X |
| C-T | 0.07 | 14X | A-T | 0.9 | — |
| C-C | 0.03 | 33X | A-C | 0.14 | 6X |
| T-G | 0.28 | 4X | G-G | 0.28 | 3X |
| T-A | 1 | — | G-A | 0.11 | 9X |
| T-T | 0.16 | 6X | G-T | 0.20 | 5X |
| T-C | 0.22 | 5X | G-C | 0.95 | — |

A similar experiment was carried out for chemical ligation, using 0.5 μM of the four FAM-labeled oligonucleotide moieties shown above (SEQ ID NOs: 7, 2, 13, and 1), with 3′-phosphorothioates, 0.5 μM of the four templates (SEQ ID NOs: 6, 4, 14, and 15), and 0.5 μM of the biotin-labeled oligonucleotide moiety (SEQ ID NO: 3) with a 5′ iodo leaving group. Each chemical ligation combination was carried out in TE buffer with 10 mM MgCl₂ at various temperatures. FAM-labeled chemically ligated products were bound to streptavidin-coated beads and analyzed on an Attune® Acoustic Focusing Cytometer as described above.

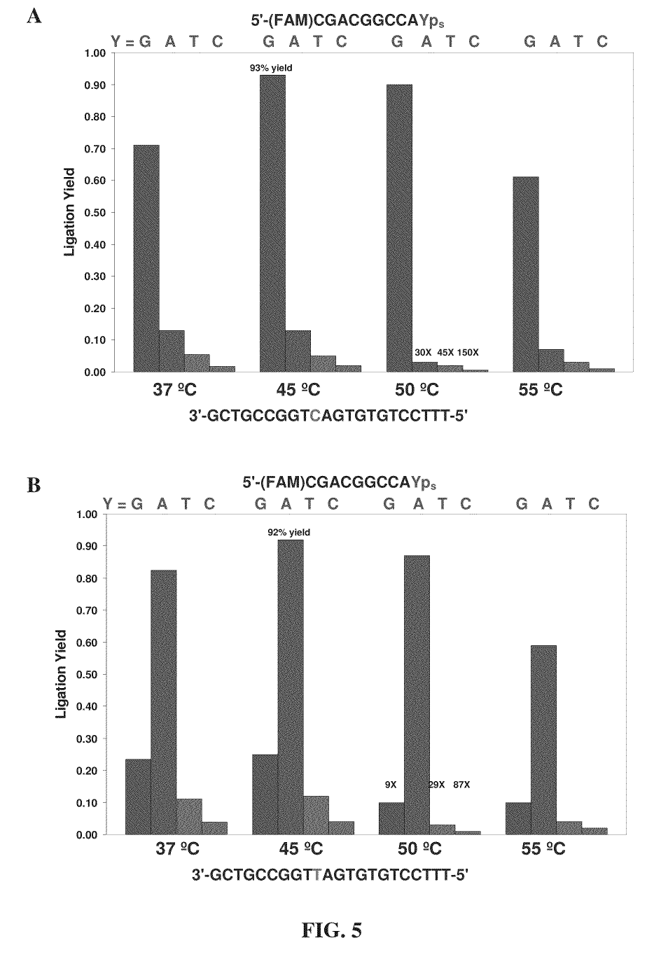

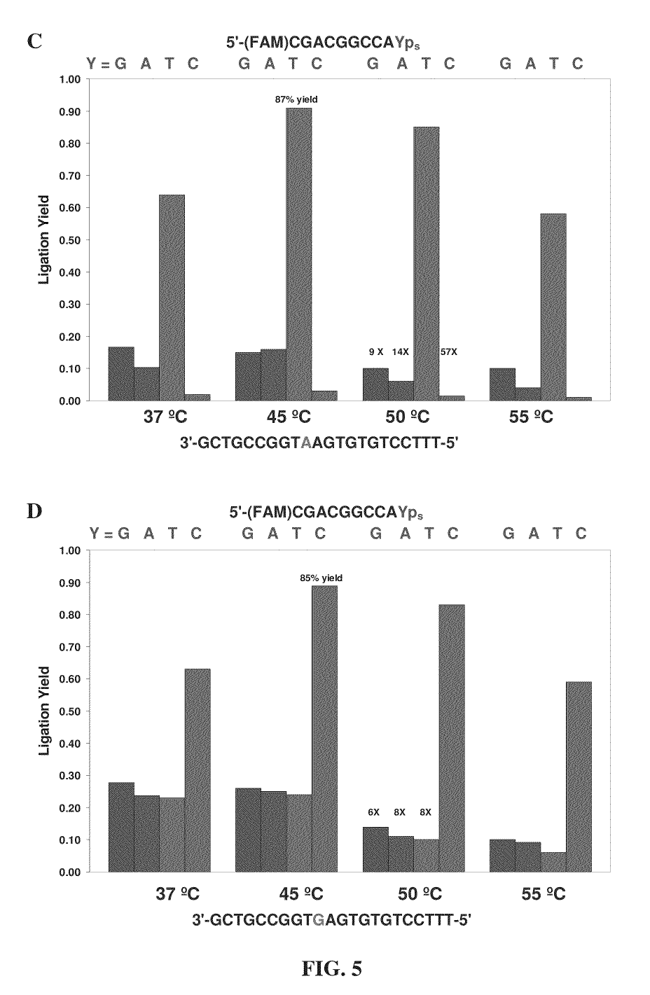

The results of that experiment are shown in FIGS. 5A to 5D. The greatest yield of ligated product (85% to 93%) was obtained at 45° C. in that experiment. Further, the greatest selectivity was obtained at 50° C. For example, as shown in FIG. 5A, the selectivity for the correct 3′ terminal nucleotide on the FAM-labeled oligonucleotide moiety with the “C” template was between 30-fold and 150-fold at 50° C. The selectivity with the other templates was somewhat lower, with the selectivity of the “T” template being between 9-fold and 87-fold, the selectivity of the “A” template being between 9-fold and 57-fold, and the selectivity of the “G” template being between 6-fold and 8-fold.

Table 2 shows the results of the chemical ligation experiment at 50° C.

TABLE 2
Ligation Yield and Selectivity for Enzymatic Ligation

| Base pair | | | Base Pair | Ligation | |
|---|---|---|---|---|---|
| (X-Y) | Ligation yield | Selectivity | (X-Y) | Yield | Selectivity |
| C-G | 0.90 | — | A-G | 0.10 | 9X |
| C-A | 0.03 | 30X | A-A | 0.06 | 14X |
| C-T | 0.02 | 45X | A-T | 0.85 | — |
| C-C | 0.006 | 150X | A-C | 0.015 | 57X |
| T-G | 0.10 | 9X | G-G | 0.14 | 6X |
| T-A | 0.87 | — | G-A | 0.11 | 8X |
| T-T | 0.03 | 29X | G-T | 0.10 | 8X |
| T-C | 0.01 | 87X | G-C | 0.83 | — |

Overall, the selectivity of chemical ligation for the correct 3′ terminal nucleotide on the FAM-labeled oligonucleotide moiety was comparable to, or greater than, the selectivity of enzymatic ligation.

### Example 4

### Amplification of Chemical Ligation

In order to determine conditions under which the chemical ligation signal is amplified, various concentrations of template and two different ligation temperatures were tested as follows. Five μM FAM-labeled oligonucleotide moiety with the sequence:

(SEQ ID NO: 2)
5′-(FAM)CGACGGCCAA-3′

and a 3′ phosphorothioate and 5 μM biotin-labeled oligonucleotide moiety with the sequence:

(SEQ ID NO: 3)
5′-TCACACAGGAAA(PEG)(PEG)(biotin)-3′

and a 5′ iodo leaving group were used for each reaction. One nM, 5 nM, or 50 nM of a DNA template with the sequence:

(SEQ ID NO: 4)
5′-TTTCCTGTGTGATTGGCCGTCG-3′

was mixed with the FAM-labeled oligonucleotide moiety and the biotin-labeled oligonucleotide moiety in TE buffer with 10 mM MgCl₂ and cycled for 10 minutes at a ligation temperature, and then 45 seconds at 90° C. for 30 cycles. The ligation temperatures tested were 37° C. and 50° C. A parallel set of reaction mixtures were maintained under isothermal conditions (37° C. or 50° C.) for the same length of time as required for the 30 cycles.

Ligated product was bound to streptavidin-coated 10 μm latex beads and free FAM-labeled oligonucleotide moieties were washed off with TE buffer at 50° C. The fluorescence intensity of FAM bound to the streptavidin-coated beads was measured by flow cytometry using a BD LSR II flow cytometer with 488 nm excitation and a 525/50 emission filter. Blank beads were used as control to set up the flow cytometer. The PMT voltage setting on the Blue-E channel (with a 525/50 band-pass filter) was adjusted so that the mean fluorescence intensity (MFI) of the blank beads was less than 50. The same instrument settings were used to run the stained beads, collecting 10,000 events for each sample.

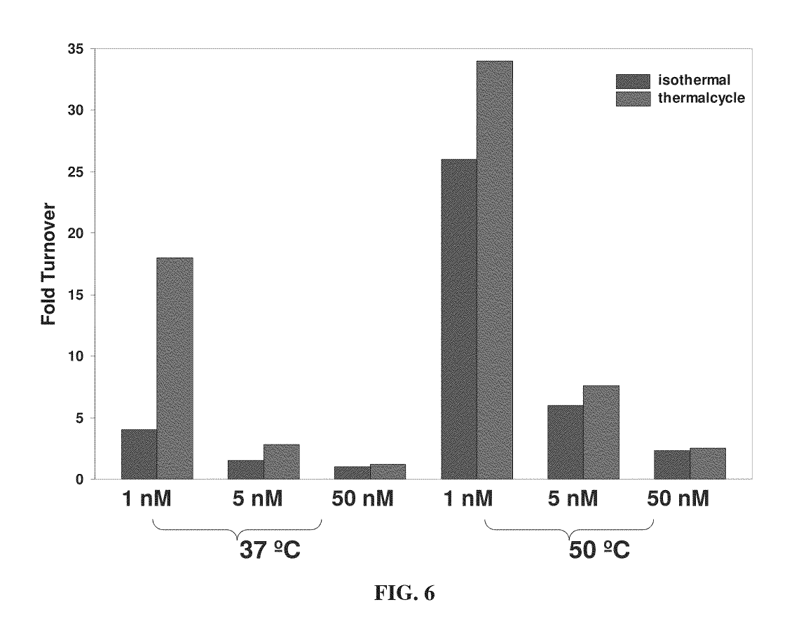

The results of that experiment are shown in FIG. 6. At 37° C., thermalcycling the chemical ligation reaction had the greatest impact at the lowest concentration of template tested, 1 nM, resulting in 18-fold turnover of chemical ligation, compared to about 4-fold turnover under isothermal conditions. The effect of thermalcycling was less pronounced at 5 nM and 50 nM at 37° C. The overall turnover was greater at 50° C., again with the greatest increase at the lowest concentration of template. At 1 nM template, thermalcycling with 50° C. ligation temperature resulted in about 34-fold turnover, compared to about 27-fold turnover under isothermal conditions. Again, the effect was less pronounced at 5 nM and 50 nM template.

Those results suggest that thermalcycling can amplify a chemical ligation signal, particularly at lower template concentrations.

### Example 5

### Single Nucleotide Polymorphism (SNP) Genotyping Using Chemical Ligation

To test the use of chemical ligation for SNP detection, a first system was designed comprising a FAM-labeled oligonucleotide moiety with the sequence:

(SEQ ID NO: 2)
5′-(FAM)CGACGGCCAA-3′

and a 3′ phosphorothioate, a VIC-labeled oligonucleotide moiety with the sequence:

(SEQ ID NO: 16)
5′-(VIC)CGACGGCCAG-3′

and a 3′ phosphorothioate, a biotin-labeled oligonucleotide moiety with the sequence:

(SEQ ID NO: 3)
5′-TCACACAGGAAA(PEG)(PEG)(biotin)-3′

and a 5′ iodo leaving group, and two templates with the sequences:

(SEQ ID NO: 4; “T” template)
5′-TTTCCTGTGTGATTGGCCGTCG-3′
and
(SEQ ID NO: 6; “C” template)
5′-TTTCCTGTGTGACTGGCCGTCG-3′.

A second system was also designed comprising a FAM-labeled oligonucleotide moiety with the sequence:

(SEQ ID NO: 13)
5′-(FAM)CGACGGCCAT-3′

and a 3′ phosphorothioate, a VIC-labeled oligonucleotide moiety with the sequence:

(SEQ ID NO: 17)
5′-(VIC)CGACGGCCAC-3′

and a 3′ phosphorothioate, a biotin-labeled oligonucleotide moiety with the sequence:

(SEQ ID NO: 3)
5′-TCACACAGGAAA(PEG)(PEG)(biotin)-3′

and a 5′ iodo leaving group, and two templates with the sequences:

(SEQ ID NO: 14; “A” template)
5′-TTTCCTGTGTGAATGGCCGTCG-3′
and
(SEQ ID NO: 15; “G” template)
5′-TTTCCTGTGTGAGTGGCCGTCG-3′.

For each system, 0.5 μm of the FAM-labeled oligonucleotide moiety, 0.5 μm of the VIC labeled oligonucleotide moiety, and 0.5 μm of the biotin-labeled oligonucleotide moiety were mixed with 0.5 μm of the first template (SEQ ID NO: 4 or 14), or 0.5 μm of the second template (SEQ ID NO: 6 or 15), or 0.25 μm of the first template and 0.25 μm of the second template. Each reaction was in TE buffer with 10 mM MgCl₂ and incubated for 2 hours at 45° C. or at 50° C. Ligated product was bound to streptavidin-coated 10 μm latex beads and free FAM-labeled oligonucleotide moieties and free VIC-labeled oligonucleotide moieties were washed off with TE buffer at 50° C. The fluorescence intensity of FAM and/or VIC bound to the streptavidin-coated beads was measured by flow cytometry using a BD LSR II flow cytometer with a 488 nm excitation and a 525/50 emission filter for FAM label, and a 532 nm excitation and a 565/20 emission filter for VIC label. Blank beads were used as control to set up the flow cytometer. The PMT voltage setting on the Blue-E channel (with a 525/50 band-pass filter) and the Green-B channel (with a 565/20 band-pass filter) was adjusted so that the mean fluorescence intensity (MFI) of the blank beads was less than 50. The same instrument settings were used to run the stained beads, collecting 10,000 events for each sample.

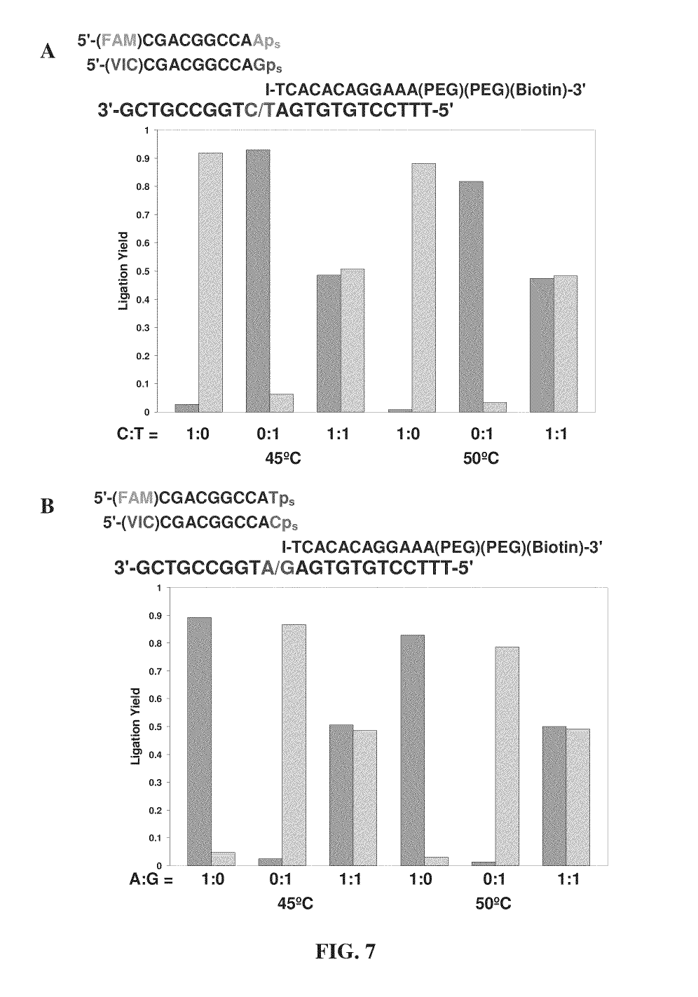

The results are shown in FIG. 7. FIG. 7A shows the results for the first system, comprising the “T” template and the “C” template. As shown in that figure, chemical ligation gave good selectivity and accurately reflected the proportions of templates in the reaction mixtures. Similar results were obtained for the second system, comprising the “A” template and the “G” template. See FIG. 7B.

### Example 6

### Labeling Solid Support Particles Using Chemical Ligation

Chemical ligation was used to label solid support particles (in this case, beads) and determine whether adjusting the ratio of the detectably labeled oligonucleotide moiety (“probe oligo”), the biotin-labeled oligonucleotide moiety (“capture oligo”), and/or the template oligonucleotide could be used to alter the relative intensity of the label on the beads, allowing multiple color and intensity labeling.

In the first experiment, the ratio of a FAM-labeled oligonucleotide moiety to a biotin-labeled oligonucleotide moiety were varied, with the concentration of template oligonucleotide kept in a constant ratio relative to the biotin-labeled oligonucleotide moiety. The FAM-labeled oligonucleotide moiety had the sequence:

(SEQ ID NO: 1)
5′-(FAM)CGACGGCCAC-3′

and a 3′ phosphorothioate; the biotin-labeled oligonucleotide moiety had the sequence:

(SEQ ID NO: 3)
5′-TCACACAGGAAA(PEG)(PEG)(biotin)-3′

and a 5′ iodo leaving group; and the template had the sequence:

(SEQ ID NO: 15)
5′-TTTCCTGTGTGAGTGGCCGTCG-3′.

Four reactions were carried out, with the concentrations of oligonucleotides shown in Table 3.

TABLE 3
Oligonucleotide Concentrations for One-Color Encoding

| Reaction | FAM-labeled oligo | Biotin-labeled oligo | Template oligo |
|---|---|---|---|
| 1:100 | 0.01 μM | 1 μM | 1 μM |
| 1:20 | 0.05 μM | 1 μM | 1 μM |
| 1:4 | 0.25 μM | 1 μM | 1 μM |
| 1.25:1 | 1.25 μM | 1 μM | 1 μM |

Each reaction was incubated in TE buffer with 10 mM MgCl₂ for 3 hours at 45° C. Ligated product was bound to streptavidin-coated 10 μm latex beads and free FAM-labeled oligonucleotide moieties were washed off with TE buffer at 50° C. The fluorescence intensity of FAM bound to the streptavidin-coated beads was measured by flow cytometry using a BD LSR II flow cytometer with 488 nm excitation and a 525/50 emission filter. Blank beads were used as control to set up the flow cytometer. The PMT voltage setting on the Blue-E channel (with a 525/50 band-pass filter) was adjusted so that the mean fluorescence intensity (MFI) of the blank beads was less than 50. The same instrument settings were used to run the stained beads, collecting 10,000 events for each sample.

The results of that experiment are shown in FIG. 8. The ratio of probe oligo to capture oligo correlated well with the amount of label observed on the beads.

In the second experiment, the ratio of template to a biotin-labeled oligonucleotide moiety were varied, with the concentration of a FAM-labeled oligonucleotide moiety kept in a constant ratio relative to the biotin-labeled oligonucleotide moiety. The same oligonucleotides were used as in the first experiment. Again, four reactions were carried out, with the concentrations of oligonucleotides shown in Table 4.

TABLE 4
Oligonucleotide Concentrations for One-Color Encoding

| Reaction | FAM-labeled oligo | Biotin-labeled oligo | Template oligo |
|---|---|---|---|
| 1:100 | 1 μM | 1 μM | 0.01 μM |
| 1:20 | 1 μM | 1 μM | 0.05 μM |
| 1:4 | 1 μM | 1 μM | 0.25 μM |
| 1.25:1 | 1 μM | 1 μM | 1.25 μM |

Each reaction was incubated in TE buffer with 10 μM MgCl₂ for 3 hours at 45° C. Ligated product was bound to streptavidin-coated 10 μm latex beads and free FAM-labeled oligonucleotide moieties were washed off with TE buffer at 50° C. The fluorescence intensity of FAM bound to the streptavidin-coated beads was measured by flow cytometry using a BD LSR II flow cytometer with 488 nm excitation and a 525/50 emission filter. Blank beads were used as control to set up the flow cytometer. The PMT voltage setting on the Blue-E channel (with a 525/50 band-pass filter) was adjusted so that the mean fluorescence intensity (MFI) of the blank beads was less than 50. The same instrument settings were used to run the stained beads, collecting 10,000 events for each sample.

The results of that experiment are shown in FIG. 9. The ratio of template to capture oligo correlated well with the amount of label observed on the beads.

These experiments demonstrated that chemical ligation can be used to label solid support particles based not only on color, but intensity.

### Example 7

### Labeling Solid Support Particles with Multiple Labels Using Chemical Ligation

Chemical ligation was used to label solid support particles (in this case, beads) with two different colors at five different ratios of the two colors. For this experiment, a FAM-labeled oligonucleotide moiety with the sequence:

(SEQ ID NO: 1)
5′-(FAM)CGACGGCCAC-3′

and a 3′ phosphorothioate; a VIC-labeled oligonucleotide moiety with the sequence:

(SEQ ID NO: 16)
5′-(VIC)CGACGGCCAG-3′

and a 3′ phosphorothioate, a biotin-labeled oligonucleotide moiety with the sequence:

(SEQ ID NO: 3)
5′-TCACACAGGAAA(PEG)(PEG)(biotin)-3′

and a 5′ iodo leaving group, and two templates with the sequences:

(SEQ ID NO: 15; “G” template)
5′-TTTCCTGTGTGAGTGGCCGTCG-3′
(SEQ ID NO: 6; “C” template)
5′-TTTCCTGTGTGACTGGCCGTCG-3′

were used. The concentration of the FAM-labeled oligonucleotide moiety, the VIC-labeled oligonucleotide moiety, and the biotin-labeled oligonucleotide moiety were kept constant, while the ratio of the templates was varied. Table 5 shows the concentrations of each oligo in each reaction.

TABLE 5
Oligonucleotide Concentrations for Two-Color Encoding

| | VIC-l | FAM- | | | |
|---|---|---|---|---|---|
| | abeled | labeled | Biotin-labeled | “G” | “C” |
| Reaction | oligo | oligo | oligo | template | template |
| 100:0 | 1 μM | 1 μM | 1 μM | 1 μM | 0 μM |
| 90:10 | 1 μM | 1 μM | 1 μM | 0.9 μM | 0.1 μM |
| 50:50 | 1 μM | 1 μM | 1 μM | 0.5 μM | 0.5 μM |
| 10:90 | 1 μM | 1 μM | 1 μM | 0.1 μM | 0.9 μM |
| 0:100 | 1 μM | 1 μM | 1 μM | 0 μM | 1 μM |

Each reaction was incubated in TE buffer with 10 μM MgCl₂ for 3 hours at 45° C. Ligated product was bound to streptavidin-coated 10 μm latex beads and free FAM-labeled oligonucleotide moieties were washed off with TE buffer at 50° C. The fluorescence intensity of FAM bound to the streptavidin-coated beads was measured by flow cytometry using a BD LSR II flow cytometer with 488 nm excitation and a 525/50 emission filter. Blank beads were used as control to set up the flow cytometer. The PMT voltage setting on the Blue-E channel (with a 525/50 band-pass filter) was adjusted so that the mean fluorescence intensity (MFI) of the blank beads was less than 50. The same instrument settings were used to run the stained beads, collecting 10,000 events for each sample.

The results of that experiment are shown in FIG. 10. The ratio of the two template oligos correlated well with the ratio of labels observed on the beads.

A system for labeling beads with four different detectable labels (Dye1, Dye2, Dye3, Dye4) is shown in FIG. 11. By adjusting the ratio of the four template oligonucleotides, different intensities of each color can be achieved. Thus, for example, using five different molar ratios of the four templates, each of which is specific for a different dye-labeled oligonucleotide moiety, 625 (or 5⁴) codes can be created.

Although the disclosed teachings have been described with reference to various applications, methods, and compositions, it will be appreciated that various changes and modifications may be made without departing from the teachings herein. The foregoing examples are provided to better illustrate the present teachings and are not intended to limit the scope of the teachings herein. Certain aspects of the present teachings may be further understood in light of the following claims.

1. A method of detecting a single nucleotide polymorphism in a target nucleic acid, comprising:
(a) contacting said target nucleic acid with a first allele-specific primer that hybridizes to a portion of the target nucleic acid comprising the single nucleotide polymorphism and a locus-specific primer, wherein the first allele-specific primer comprises a 3′ nucleophile, and the locus-specific primer comprises a 5′ leaving group, wherein the first allele specific primer and the locus-specific primer hybridize to the target nucleic acid such that the 5′ end of the locus-specific primer is adjacent to the 3′ end of the first allele-specific primer, under conditions allowing chemical ligation between the first allele-specific primer and the first locus-specific primer to form a ligated product; and
(b) detecting the ligated product.

2. The method of claim 1, wherein the method further comprises contacting the target nucleic acid with a second allele-specific primer that hybridizes to a portion of the target nucleic acid comprising the single nucleotide polymorphism, wherein the second allele-specific primer comprises a 3′ nucleophile, wherein the second allele-specific primer differs from the first allele-specific primer at least at the nucleotide that hybridizes with the single nucleotide polymorphism, and wherein the second allele specific primer and the locus-specific primer hybridize to the target nucleic acid such that the 5′ end of the locus-specific primer is adjacent to the 3′ end of the second allele-specific primer.

3. A method of detecting a single nucleotide polymorphism in a target nucleic acid, comprising:
(a) contacting the target nucleic acid with a first allele-specific primer that hybridizes to a portion of the target nucleic acid comprising the single nucleotide polymorphism and a locus-specific primer, wherein the first allele-specific primer comprises a 5′ leaving group, and the locus-specific primer comprises a 3′ nucleophile, wherein the first allele-specific primer and the locus-specific primer hybridize to the target nucleic acid such that the 5′ end of the first allele-specific primer is adjacent to the 3′ end of the locus-specific primer, under conditions allowing chemical ligation between the first allele-specific primer and the locus-specific primer to form a ligated product; and
(b) detecting the ligated product.

4. The method of claim 3, wherein the method further comprises contacting the target nucleic acid with a second allele-specific primer that hybridizes to a portion of the target nucleic acid comprising the single nucleotide polymorphism, wherein the second allele-specific primer comprises a 5′ leaving group, wherein the second allele-specific primer differs from the first allele-specific primer at least at the nucleotide that hybridizes with the single nucleotide polymorphism, and wherein the second allele-specific primer and the locus-specific primer hybridize to the target nucleic acid such that the 5′ end of the second allele-specific primer is adjacent to the 3′ end of the locus-specific primer.

5. The method of any one of the preceding claims, wherein the locus-specific primer comprises a sequence that is complementary to between 3 and 60 contiguous nucleotides of the target nucleic acid.

6. The method of any one of the preceding claims, wherein the first allele-specific primer comprises a sequence that is complementary to between 3 and 60 contiguous nucleotides of the target nucleic acid.

7. The method of claim 2 or claim 4, wherein the second allele-specific primer comprises a sequence that is complementary to between 3 and 60 contiguous nucleotides of the target nucleic acid.

8. The method of any one of the preceding claims, wherein detecting the ligated product comprises enzymatically amplifying the ligated product.

9. The method of claim 8, wherein detecting the ligated product comprises enzymatically amplifying the ligated product using one or more of real-time PCR, qPCR, and digital PCR.

10. The method of claim 8 or claim 9, wherein the first allele-specific primer comprises a first portion that is complementary to the target nucleic acid and a second portion that is complementary to a first amplification primer, and the locus-specific primer comprises a first portion that is complementary to the target nucleic acid and a second portion that is complementary to a second amplification primer, and wherein detecting the ligated product comprises enzymatically amplifying the ligated product in the presence of the first amplification primer and the second amplification primer.

11. The method of any one of claims 1 to 7, wherein the first allele-specific primer comprises a detectable label, or the locus-specific primer comprises a detectable label, or the first allele-specific primer comprises a first detectable label and the locus-specific primer comprises a second detectable label, wherein the first and second detectable labels are the same or different.

12. A method of detecting at least one target analyte in a sample, comprising:
(a) contacting the at least one target analyte with:
(i) a first proximity detection probe comprising a first analyte binding moiety and a first oligonucleotide moiety, wherein the first oligonucleotide moiety comprises a 3′ nucleophile;
(ii) a second proximity detection probe comprising a second analyte binding moiety and a second oligonucleotide moiety, wherein the second oligonucleotide moiety comprises a 5′ leaving group; and
(iii) a splint oligonucleotide comprising a first portion that hybridizes with a portion of the first oligonucleotide moiety and a second portion that hybridizes with the second oligonucleotide moiety such that the 3′ end of the first oligonucleotide moiety is adjacent to the 5′ end of the second oligonucleotide moiety;
under conditions allowing formation of a complex comprising at least one target analyte, the first proximity detection probe, the second proximity detection probe, and the splint oligonucleotide, and allowing chemical ligation between the first oligonucleotide moiety and the second oligonucleotide moiety to form a ligated product; and
(b) detecting the ligated product.

13. The method of claim 12, wherein the method comprises removing unbound first proximity detection probe, removing unbound second proximity detection probe, or removing unbound first proximity detection probe and removing unbound second proximity detection probe.

14. The method of claim 12, wherein the target analyte is selected from a protein, a peptide, a carbohydrate, and a hormone.

15. The method of claim 14, wherein the target analyte is a protein.

16. The method of claim 12, wherein the first analyte binding moiety and the second analyte binding moiety are capable of binding to the same target analyte.

17. The method of claim 12, wherein the first analyte binding moiety and the second analyte binding moiety are capable of binding to different target analytes.

18. The method of claim 12, wherein at least one of the analyte binding moieties is a covalent analyte binding moiety.

19. The method of claim 18, wherein the covalent analyte binding moiety is capable of covalently attaching to an enzyme selected from a metalloprotease, a cysteine protease, a ubiquitin-specific protease, a cysteine cathepsin, an esterase, a kinase, a histone deacetylase, a serine reductase, an oxidoreductase, an ATPase, and a GTPase.

20. The method of claim 12, wherein at least one of the analyte binding moieties is a noncovalent analyte binding moiety.

21. The method of claim 20, wherein the noncovalent analyte binding moiety is selected from an antibody, a protein, a peptide, a lectin, a nucleic acid, an aptamers, a carbohydrate, a soluble receptor, and a small molecule.

22. The method of any one of claims 12 to 21, wherein the detecting comprises enzymatically amplifying the ligated product.

23. The method of claim 22, wherein the detecting comprises enzymatically amplifying the ligated product using one or more of real-time PCR, qPCR, and digital PCR.

24. A method of labeling a solid support particle, comprising contacting a solid support particle comprising a first member of a binding pair, with:
(i) a first oligonucleotide moiety comprising a 3′ nucleophile, and further comprising a second member of a binding pair;
(ii) a second oligonucleotide moiety comprising a 5′ leaving group, and further comprising at least one detectable label; and
(iii) a splint oligonucleotide comprising a first portion that hybridizes with a portion of the first oligonucleotide moiety and a second portion that hybridizes with the second oligonucleotide moiety such that the 3′ end of the first oligonucleotide moiety is adjacent to the 5′ end of the second oligonucleotide moiety;
under conditions allowing binding of the first member of the binding pair to the second member of the binding pair, and allowing formation of a complex comprising the solid support particle, the first oligonucleotide moiety, the second oligonucleotide moiety, and the splint oligonucleotide, and allowing chemical ligation between the first oligonucleotide moiety and the second oligonucleotide moiety.

25. A method of labeling a solid support particle, comprising combining a solid support particle comprising a first member of a binding pair, with:
(i) a first oligonucleotide moiety comprising a 5′ leaving group, and further comprising a second member of a binding pair;
(ii) a second oligonucleotide moiety comprising a 3′ nucleophile, and further comprising at least one detectable label; and
(iii) a splint oligonucleotide comprising a first portion that hybridizes with a portion of the first oligonucleotide moiety and a second portion that hybridizes with the second oligonucleotide moiety such that the 5′ end of the first oligonucleotide moiety is adjacent to the 3′ end of the second oligonucleotide moiety;
under conditions allowing binding of the first member of the binding pair to the second member of the binding pair, and allowing formation of a complex comprising the solid support particle, the first oligonucleotide moiety, the second oligonucleotide moiety, and the splint oligonucleotide, and allowing chemical ligation between the first oligonucleotide moiety and the second oligonucleotide moiety.

26. The method of claim 24 or claim 25, wherein the ratio of second oligonucleotide to first oligonucleotide is between 10:1 and 1:200.

27. The method of claim 26, wherein the ratio is between 5:1 and 1:100.

28. The method of claim 27, wherein the ratio is between 2:1 and 1:50.

29. The method of claim 24 or claim 25, wherein the ratio of splint oligonucleotide and first oligonucleotide is between 10:1 and 1:200.

30. The method of claim 29, wherein the ratio is between 5:1 and 1:100.

31. The method of claim 30, wherein the ratio is between 2:1 and 1:50.

32. A method of labeling a solid support particle, comprising contacting a solid support particle comprising a first member of a binding pair, with:
(i) a first oligonucleotide moiety comprising a 3′ nucleophile, and further comprising a second member of a binding pair;
(ii) a second oligonucleotide moiety comprising a 5′ leaving group, and further comprising a first detectable label;
(iii) a third oligonucleotide moiety comprising a 5′ leaving group, and further comprising a second detectable label;
(iv) a first splint oligonucleotide comprising a first portion that hybridizes with a portion of the first oligonucleotide moiety and a second portion that hybridizes with the second oligonucleotide moiety such that the 3′ end of the first oligonucleotide moiety is adjacent to the 5′ end of the second oligonucleotide moiety; and
(v) a second splint oligonucleotide comprising a first portion that hybridizes with a portion of the first oligonucleotide moiety and a second portion that hybridizes with the third oligonucleotide moiety such that the 3′ end of the first oligonucleotide moiety is adjacent to the 5′ end of the third oligonucleotide moiety;
under conditions allowing binding of the first member of the binding pair to the second member of the binding pair, and allowing formation of a first complex comprising the solid support particle, the first oligonucleotide moiety, the second oligonucleotide moiety, and the first splint oligonucleotide, and a second complex comprising the solid support particle, the first oligonucleotide moiety, the third oligonucleotide moiety, and the second splint oligonucleotide, and allowing chemical ligation between the first oligonucleotide moiety and the second oligonucleotide moiety, and between the first oligonucleotide moiety and the third oligonucleotide moiety.

33. The method of claim 32, wherein the ratio of first splint oligonucleotide to second splint oligonucleotide is between 500:1 and 1:500.

34. The method of claim 33, wherein the ratio of first splint oligonucleotide to second splint oligonucleotide is between 100:1 and 1:100.

35. The method of claim 34, wherein the ratio of first splint oligonucleotide to second splint oligonucleotide is between 10:1 and 1:10.

36. The method of claim 32, wherein the method further comprises combining the solid particle comprising a first member of a binding pair with:
(vi) a fourth oligonucleotide moiety comprising a 5′ leaving group, and further comprising a third detectable label; and
(vii) a third splint oligonucleotide comprising a first portion that hybridizes with a portion of the first oligonucleotide moiety and a second portion that hybridizes with the fourth oligonucleotide moiety such that the 3′ end of the first oligonucleotide moiety is adjacent to the 5′ end of the fourth oligonucleotide moiety;
under conditions allowing binding of the first member of the binding pair to the second member of the binding pair, and allowing formation of a complex comprising the solid support particle, the first oligonucleotide moiety, the fourth oligonucleotide moiety, and the third splint oligonucleotide, and allowing chemical ligation between the first oligonucleotide moiety and the fourth oligonucleotide moiety.

37. The method of claim 36, wherein the method further comprises combining the solid particle comprising a first member of a binding pair with:
(vi) a fifth oligonucleotide moiety comprising a 5′ leaving group, and further comprising a fourth detectable label; and
(vii) a fourth splint oligonucleotide comprising a first portion that hybridizes with a portion of the first oligonucleotide moiety and a second portion that hybridizes with the fifth oligonucleotide moiety such that the 3′ end of the first oligonucleotide moiety is adjacent to the 5′ end of the fifth oligonucleotide moiety;
under conditions allowing binding of the first member of the binding pair to the second member of the binding pair, and allowing formation of a complex comprising the solid support particle, the first oligonucleotide moiety, the fifth oligonucleotide moiety, and the fourth splint oligonucleotide, and allowing chemical ligation between the first oligonucleotide moiety and the fifth oligonucleotide moiety.

38. A method of labeling a solid support particle, comprising contacting a solid support particle comprising a first member of a binding pair, with:
(i) a first oligonucleotide moiety comprising a 5′ leaving group, and further comprising a second member of a binding pair;
(ii) a second oligonucleotide moiety comprising a 3′ nucleophile, and further comprising a first detectable label;
(iii) a third oligonucleotide moiety comprising a 3′ nucleophile, and further comprising a second detectable label;
(iv) a first splint oligonucleotide comprising a first portion that hybridizes with a portion of the first oligonucleotide moiety and a second portion that hybridizes with the second oligonucleotide moiety such that the 5′ end of the first oligonucleotide moiety is adjacent to the 3′ end of the second oligonucleotide moiety; and
(v) a second splint oligonucleotide comprising a first portion that hybridizes with a portion of the first oligonucleotide moiety and a second portion that hybridizes with the third oligonucleotide moiety such that the 5′ end of the first oligonucleotide moiety is adjacent to the 3′ end of the third oligonucleotide moiety;
under conditions allowing binding of the first member of the binding pair to the second member of the binding pair, and allowing formation of a first complex comprising the solid support particle, the first oligonucleotide moiety, the second oligonucleotide moiety, and the first splint oligonucleotide, and a second complex comprising the solid support particle, the first oligonucleotide moiety, the third oligonucleotide moiety, and the second splint oligonucleotide, and allowing chemical ligation between the first oligonucleotide moiety and the second oligonucleotide moiety, and between the first oligonucleotide moiety and the third oligonucleotide moiety.

39. The method of claim 38, wherein the ratio of first splint oligonucleotide to second splint oligonucleotide is between 500:1 and 1:500.

40. The method of claim 39, wherein the ratio of first splint oligonucleotide to second splint oligonucleotide is between 100:1 and 1:100.

41. The method of claim 40, wherein the ratio of first splint oligonucleotide to second splint oligonucleotide is between 10:1 and 1:10.

42. The method of claim 38, wherein the method further comprises combining the solid particle comprising a first member of a binding pair with:
(vi) a fourth oligonucleotide moiety comprising a 3′ nucleophile, and further comprising a third detectable label; and
(vii) a third splint oligonucleotide comprising a first portion that hybridizes with a portion of the first oligonucleotide moiety and a second portion that hybridizes with the fourth oligonucleotide moiety such that the 5′ end of the first oligonucleotide moiety is adjacent to the 3′ end of the fourth oligonucleotide moiety;
under conditions allowing binding of the first member of the binding pair to the second member of the binding pair, and allowing formation of a complex comprising the solid support particle, the first oligonucleotide moiety, the fourth oligonucleotide moiety, and the third splint oligonucleotide, and allowing chemical ligation between the first oligonucleotide moiety and the fourth oligonucleotide moiety.

43. The method of claim 42, wherein the method further comprises combining the solid particle comprising a first member of a binding pair with:
(vi) a fifth oligonucleotide moiety comprising a 3′ nucleophile, and further comprising a fourth detectable label; and
(vii) a fourth splint oligonucleotide comprising a first portion that hybridizes with a portion of the first oligonucleotide moiety and a second portion that hybridizes with the fifth oligonucleotide moiety such that the 5′ end of the first oligonucleotide moiety is adjacent to the 3′ end of the fifth oligonucleotide moiety;
under conditions allowing binding of the first member of the binding pair to the second member of the binding pair, and allowing formation of a complex comprising the solid support particle, the first oligonucleotide moiety, the fifth oligonucleotide moiety, and the fourth splint oligonucleotide, and allowing chemical ligation between the first oligonucleotide moiety and the fifth oligonucleotide moiety.

44. The method of any one of claims 24 to 43, wherein the first member of the binding pair is a biotin-binding moiety and the second member of the binding pair is biotin or a biotin derivative.

45. The method of claim 44, wherein the biotin-binding moiety is avidin or streptavidin.

46. The method of any one of the preceding claims, wherein the 3′ nucleophile is selected from phosphorothioate, phosphoroselenoate, phosphorotelluroate, thiol, thiocarboxylate, dithiocarboxylate, amino, hydrazine, hydroxylamine, selenol, selenocarboxylate, and diselenocarboxylate.

47. The method of claim 46, wherein the 3′ nucleophile is selected from phosphorothioate, phosphoroselenoate, and phosphorotelluroate.

48. The method of any one of the preceding claims, wherein the 5′ leaving group is selected from I, Br, Cl, mesylate, tosylate, brosylate, para-nitrobenzenesulfonate, trifluoromethanesulfonate, trifluoroethanesulfonate, nonafluorobutanesulfonate, trifluoroacetate, a sulfonium cation, and a quaternary ammonium cation.

49. The method of claim 48, wherein the 5′ leaving group is selected from I, Br, and tosylate.

50. A kit comprising a first allele-specific primer and a locus-specific primer, wherein the first allele-specific primer comprises a 3′ nucleophile, and the locus-specific primer comprises a 5′ leaving group, wherein the first allele specific primer hybridizes to a portion of a target nucleic acid comprising a single nucleotide polymorphism, and wherein the first allele-specific primer and the locus-specific primer hybridize to a target nucleic acid such that the 5′ end of the locus-specific primer is adjacent to the 3′ end of the first allele-specific primer.

51. The kit of claim 50, wherein the kit further comprises a second allele-specific primer, wherein the second allele-specific primer comprises a 3′ nucleophile, wherein the second allele-specific primer differs from the first allele-specific primer at least at the nucleotide that hybridizes with the single nucleotide polymorphism, and wherein the second allele specific primer and the locus-specific primer hybridize to the target nucleic acid such that the 5′ end of the locus-specific primer is adjacent to the 3′ end of the second allele-specific primer.

52. A kit comprising a first allele-specific primer and a locus-specific primer, wherein the first allele-specific primer comprises a 5′ leaving group, and the locus-specific primer comprises a 3′ nucleophile, wherein the first allele specific primer hybridizes to a portion of a target nucleic acid comprising a single nucleotide polymorphism, and wherein the first allele-specific primer and the locus-specific primer hybridize to a target nucleic acid such that the 3′ end of the locus-specific primer is adjacent to the 5′ end of the first allele-specific primer.

53. The kit of claim 50, wherein the kit further comprises a second allele-specific primer, wherein the second allele-specific primer comprises a 5′ leaving group, wherein the second allele-specific primer differs from the first allele-specific primer at least at the nucleotide that hybridizes with the single nucleotide polymorphism, and wherein the second allele specific primer and the locus-specific primer hybridize to the target nucleic acid such that the 3′ end of the locus-specific primer is adjacent to the 5′ end of the second allele-specific primer.

54. A kit comprising a first proximity detection probe comprising a first analyte binding moiety and a first oligonucleotide moiety, wherein the first oligonucleotide moiety comprises a 3′ nucleophile; and a second proximity detection probe comprising a second analyte binding moiety and a second oligonucleotide moiety, wherein the second oligonucleotide moiety comprises a 5′ leaving group.

55. The kit of claim 54, wherein the kit further comprises a splint oligonucleotide comprising a first portion that hybridizes with a portion of the first oligonucleotide moiety and a second portion that hybridizes with the second oligonucleotide moiety such that the 3′ end of the first oligonucleotide moiety is adjacent to the 5′ end of the second oligonucleotide moiety.

56. The kit of claim 55, wherein the first analyte binding moiety and the second analyte binding moiety are capable of binding to the same target analyte.

57. The kit of claim 55, wherein the first analyte binding moiety and the second analyte binding moiety are capable of binding to different target analytes.

58. The kit of any one of claims 55 to 57, wherein at least one of the analyte binding moieties is a covalent analyte binding moiety.

59. The kit of claim 58, wherein the covalent analyte binding moiety is capable of covalently attaching to an enzyme selected from a metalloprotease, a cysteine protease, a ubiquitin-specific protease, a cysteine cathepsin, an esterase, a kinase, a histone deacetylase, a serine reductase, an oxidoreductase, an ATPase, and a GTPase.

60. The kit of any one of claims 55 to 59, wherein at least one of the analyte binding moieties is a noncovalent analyte binding moiety.

61. The kit of claim 60, wherein the noncovalent analyte binding moiety is selected from an antibody, a protein, a peptide, a lectin, a nucleic acid, an aptamers, a carbohydrate, a soluble receptor, and a small molecule.

62. The kit of any one of claims 50 to 61, wherein the 3′ nucleophile is selected from phosphorothioate, phosphoroselenoate, phosphorotelluroate, thiol, thiocarboxylate, dithiocarboxylate, amino, hydrazine, hydroxylamine, selenol, selenocarboxylate, and diselenocarboxylate.

63. The kit of claim 62, wherein the 3′ nucleophile is selected from phosphorothioate, phosphoroselenoate, and phosphorotelluroate.

64. The kit of any one of claims 50 to 63, wherein the 5′ leaving group is selected from I, Br, Cl, mesylate, tosylate, brosylate, para-nitrobenzenesulfonate, trifluoromethanesulfonate, trifluoroethanesulfonate, nonafluorobutanesulfonate, trifluoroacetate, a sulfonium cation, and a quaternary ammonium cation.

65. The kit of claim 64, wherein the 5′ leaving group is selected from I, Br, and tosylate.
